(19) United States
(12) Patent Application Publication
(10) Pub. No.: US 2026/0217844 A1
(43) Pub. Date: Jul. 30, 2026

(54) COMBINATION OF ANTIBODIES SPECIFICALLY RECOGNIZING TNFR2 AND PD-L1 OR PD-1

(71) Applicant: STAIDSON BIOPHARMA INC., Richmond, CA (US)

(72) Inventors: Zuoan YI, Mountain View, CA (US); Wenwu ZHAI, Richmond, CA (US); Chong HE, Richmond, CA (US)

(73) Assignee: STAIDSON BIOPHARMA INC., Richmond, CA (US)

(21) Appl. No.: 19/146,022

(22) PCT Filed: Jan. 5, 2024
(86) PCT No.: PCT/US2024/010381

§ 371 Date: Jul. 7, 2025

Related U.S. Application Data
(60) Provisional application No. 63/478,903, filed on Jan. 6, 2023.

(51) Int. Cl. C07K 16/28; A61K 39/00; A61P 35/00

(52) U.S. Cl. CPC C07K 16/2878; A61P 35/00; C07K 16/2818; C07K 16/2827; A61K 2039/507; C07K 2317/21; C07K 2317/24; C07K 2317/56; C07K 2317/565; C07K 2317/92

(57) ABSTRACT

The present application provides anti-TNFR2 antibodies, the pharmaceutical composition or combination comprising the anti-TNFR2 antibody and anti-PD-L1 antibody or anti-PD-1 antibody, and methods of using the pharmaceutical composition or the combination with the anti-TNFR2 antibody and anti-PD-L1 antibody or anti-PD-1 antibody to treat cancer or infectious disease.

### SUBMISSION OF SEQUENCE LISTING ON ASCII TEXT FILE

The contents of the electronic sequence listing (Combination therapy SEQLIST.xml; Size: 44 kb; and Date of Creation: Dec. 25, 2023) are herein incorporated by reference in its entirety.

### FIELD OF THE APPLICATION

This invention relates to the combination therapy of the antibody specifically recognizing human TNFR2 and the antibody specifically recognizing human PD-1 or PD-L1, as well as methods of manufacture and uses thereof, including methods of treating and preventing cancer or infectious diseases.

### BACKGROUND OF THE APPLICATION

Tumor necrosis factor receptor 2 (TNFR2), also known as tumor necrosis factor receptor superfamily member 1B (TNFRSF1B) and CD120b, is a membrane receptor that binds with cognate ligand TNFα and also with lymphotoxin-α (LTα). In contrast to TNFR1 which has a death domain (DD) in its cytoplasmic part and activates caspase-dependent pathway and NFκB pathway, TNFR2 lacks DD but can recruit the adapter protein TNF receptor associated factor 2 (TRAF2) and TRAF3 and activates the nonconical NFκB pathway and MAP kinase pathway (Brenner et al., 2015). TNFR2 is expressed on the immune cells and some non-immune cells including endothelial cells, cardiomyocytes, astrocytes, etc. (Ward-Kavanagh et al., 2016). Although early studies showed that TNFR2 co-stimulates naïve T cell function, it was later demonstrated that TNFR2 also limits CD8+ T-cell-mediated viral clearance and anti-tumor immunity by inducing rapid contraction of CD8+ T cells (Bertrand et al., 2015; DeBerge et al., 2015; Kim et al., 2009; Wortzman et al., 2013b). Several studies showed that TNFR2 expression is higher in regulatory T cells (Treg cells) than in naïve T cells and TNFR2 signaling is important for the development, proliferation, and survival of Treg cells (Chen et al., 2013; Horwitz et al., 2013; Mahmud et al., 2014). Therefore, TNFR2 signaling plays critical roles in regulating immune response. In addition, TNFR2 is highly expressed in Treg cells and myeloid-derived suppressive cells (MDSC) in tumor microenvironment, indicating the potential function of TNFR2 in tumor immunity (Chen et al., 2013; Hu et al., 2014). In fact, several publications reported the antitumor efficacy of anti-mouse TNFR2 antibody, although the mechanisms remain ill-defined (Case et al., 2020; Nie et al., 2016; Tam et al., 2019; Williams et al., 2018). Furthermore, an identified mutation in TNFR2 was linked to T cell lymphoma, including mycosis fungoides and Sezary syndrome, suggesting it may serve as an oncogene (Ungewickell et al., 2015). There are also reports showing that TNFR2 is upregulated in certain types of cancer and related with poor prognosis (Yang et al., 2017; Zhang et al., 2018).

The protein Programmed Death 1 (PD-1) is an inhibitory member of the CD28 family of receptors, which also includes CD28, CTLA-4, ICOS and BTLA. PD-1 is expressed on activated B cells, T cells, and myeloid cells (Agata et al., supra; Okazaki et al. (2002) Curr. Opin. Immunol 14:391779-82; Bennett et al. (2003) J Immunol 170:711-8). PD-1 was discovered through screening for differential expression in apoptotic cells (Ishida et al. (1992) EMBO J 11:3887-95).

PD-L1 is a B7 family member that is expressed on many cell types, including APCs and activated T cells (Yamazaki et al. (2002) J. Immunol. 169:5538). PD-L1 binds to both PD-1 and B7-1. Both binding of T-cell-expressed B7-1 by PD-L1 and binding of T-cell-expressed PD-L1 by B7-1 result in T cell inhibition (Butte et al. (2007) Immunity 27:111). There is also evidence that, like other B7 family members, PD-L1 can also provide costimulatory signals to T cells (Subudhi et al. (2004) J. Clin. Invest. 113:694; Tamura et al. (2001) Blood 97:1809). PD-L1 that is a ligand of PD-1 is expressed in so-called antigen-presenting cells (APCs) such as activated monocytes and dendritic cells (Journal of Experimental Medicine (2000), vol. 19, issue 7, p 1027-1034). These cells present interaction molecules that induce a variety of immuno-inductive signals to T lymphocytes, and PD-L1 is one of these molecules that induce the inhibitory signal by PD-1. It has been revealed that PD-L1 ligand stimulation suppressed the activation (cellular proliferation and induction of various cytokine production) of PD-1 expressing T lymphocytes. PD-L1 expression has been confirmed in not only immunocompetent cells but also a certain kind of tumor cell lines (cell lines derived from monocytic leukemia, cell lines derived from mast cells, cell lines derived from hepatic carcinomas, cell lines derived from neuroblasts, and cell lines derived from breast carcinomas) (Nature Immunology (2001), vol. 2, issue 3, p. 261-267).

In the past few years, tumor immunotherapy has become a major hot spot in the field of tumor therapy research as a new treatment modality. Antagonistic antibodies that target immune checkpoint proteins, such as anti-PD-1 and anti-PD-L1 antibodies, have been used to treat various types of cancer with revolutionary results, greatly prolonging the survival of patients with malignant tumors.

However, as tumors often utilize multiple, overlapping and redundant mechanisms to block anti-tumor immune response, combination therapy will likely be required for durable efficacy across a wide range of tumor types. Therefore, new combination therapies are needed to improve the treatment of all cancers. The inventors have prepared anti-TNFR2 antibodies and acquired better anti-tumor effects and long-lasting immune memory in combination with existing anti-PD-L1 or anti-PD-1 antibodies.

The disclosures of all publications, patents, patent applications and published patent applications referred to herein are hereby incorporated herein by reference in their entirety.

### BRIEF SUMMARY OF THE APPLICATION

In one aspect, there is provided a method of treating cancer or infectious disease in an individual in need thereof, comprising administering to the individual an antibody specifically recognizing human TNFR2 and an antibody specifically recognizing human PD-L1 or PD-1. In some embodiments, the antibody specifically recognizing human TNFR2 and the antibody specifically recognizing human PD-L1 or PD-1 are administered to the individual in need thereof concurrently or sequentially.

In one aspect, the antibody specifically recognizing human TNFR2 and the antibody specifically recognizing human PD-L1 or PD-1 for use in the treatment of cancer or infectious disease in an individual in need thereof is provided. In another aspect, the antibody specifically recognizing human TNFR2 for use in the combined treatment of cancer or infectious disease with the antibody specifically recognizing human PD-L1 or PD-1 in an individual in need thereof. In some embodiments, the antigen-binding specifically recognizing human TNFR2 and an antibody specifically recognizing human PD-L1 or PD-1 are administered to the individual in need thereof concurrently or sequentially.

In some embodiments, the method or the antibody for use described above, wherein, the administration to the individual an antibody specifically recognizing human TNFR2 is during the period of treatment with an antibody specifically recognizing human PD-L1 or PD-1.

In some embodiments, the method or the antibody for use described above, wherein, the administration to the individual an antibody specifically recognizing human PD-L1 or PD-1 is during the period of treatment with an antibody specifically recognizing human TNFR2.

In another aspect, the present application provided a pharmaceutical composition comprising an antibody specifically recognizing human TNFR2 and an antibody specifically recognizing human PD-L1 or PD-1.

In another aspect, the present application provided a combination of an antibody specifically recognizing human TNFR2 and an antibody specifically recognizing human PD-L1 or PD-1.

In some embodiments, there is provided a method of treating cancer or infectious disease in an individual in need thereof comprising administering to the individual a pharmaceutical composition or the combination described above.

In some embodiments, the pharmaceutical composition for use in the treatment of cancer or infectious disease in an individual in need thereof is provided.

In some embodiments, there is provided a method of treating cancer or infectious disease in an individual in need thereof comprising administering to the individual the combination described above, wherein the antibody specifically recognizing human TNFR2 and the antibody specifically recognizing human PD-L1 or PD-1 are administrated to the individual in need thereof concurrently or sequentially.

In some embodiments, the combination for use in the treatment of cancer or infectious disease in an individual in need thereof, wherein the antibody specifically recognizing human TNFR2 and the antibody specifically recognizing human PD-L1 or PD-1 are administrated to the individual in need thereof concurrently or sequentially.

In some embodiments, the antibody specifically recognizing the human TNFR2 described above comprises a V_{H} comprising an HC-CDR1 comprising the amino acid sequence SEQ ID NO: 1, an HC-CDR2 comprising the amino acid sequence of SEQ ID NO: 2, and an HC-CDR3 comprising the amino acid sequence of SEQ ID NO: 3; and a V_{L} comprising: an LC-CDR1 comprising the amino acid sequence of SEQ ID NO: 4, an LC-CDR2 comprising the amino acid sequence of SEQ ID NO: 5, and an LC-CDR3 comprising the amino acid sequence of SEQ ID NO: 6.

In some embodiments, the antibody specifically recognizing the human TNFR2 comprises a V_{H} comprising an HC-CDR1, an HC-CDR2, and an HC-CDR3 of a V_{H} comprising the amino acid sequence of any one of SEQ ID NOs: 7-15; and a V_{L} comprising a LC-CDR1, a LC-CDR2, and a LC-CDR3 of a V_{L} comprising the amino acid sequence of any one of SEQ ID NOs: 16-20.

In some embodiments, the antibody specifically recognizing the human TNFR2 comprises a V_{H} comprising the amino acid sequence of any one of SEQ ID NOs: 7-15 or a variant thereof having at least about 80% sequence identity to the amino acid sequence of any one of SEQ ID NOs: 7-15, and a V_{L} comprising the amino acid sequence of any one of SEQ ID NOs: 16-20, or a variant thereof having at least about 80% sequence identity to the amino acid sequence of any one of SEQ ID NOs: 16-20.

In some embodiments, the antibody specifically recognizing the human TNFR2 comprises a V_{H} comprising the amino acid sequence of SEQ ID NO: 7, or a variant thereof having at least about 80% sequence identity to the amino acid sequence of SEQ ID NO: 7; and a V_{L} comprising the amino acid sequence of SEQ ID NO: 16, or a variant thereof having at least about 80% sequence identity to the amino acid sequence of SEQ ID NO: 16.

In some embodiments, the antibody specifically recognizing the human TNFR2 comprises a V_{H} comprising the amino acid sequence of SEQ ID NO: 8, or a variant thereof having at least about 80% sequence identity to the amino acid sequence of SEQ ID NO: 8; and a V_{L} comprising the amino acid sequence of SEQ ID NO: 17, or a variant thereof having at least about 80% sequence identity to the amino acid sequence of SEQ ID NO: 17.

In some embodiments, the antibody specifically recognizing the human TNFR2 comprises a V_{H} comprising the amino acid sequence of SEQ ID NO: 9, or a variant thereof having at least about 80% sequence identity to the amino acid sequence of SEQ ID NO: 9; and a V_{L} comprising the amino acid sequence of SEQ ID NO: 18, or a variant thereof having at least about 80% sequence identity to the amino acid sequence of SEQ ID NO: 18.

In some embodiments, the antibody specifically recognizing the human TNFR2 comprises a V_{H} comprising the amino acid sequence of SEQ ID NO: 9, or a variant thereof having at least about 80% sequence identity to the amino acid sequence of SEQ ID NO: 9; and a V_{L} comprising the amino acid sequence of SEQ ID NO: 19, or a variant thereof having at least about 80% sequence identity to the amino acid sequence of SEQ ID NO: 19.

In some embodiments, the antibody specifically recognizing the human TNFR2 comprises a V_{H} comprising the amino acid sequence of SEQ ID NO: 9, or a variant thereof having at least about 80% sequence identity to the amino acid sequence of SEQ ID NO: 9; and a V_{L} comprising the amino acid sequence of SEQ ID NO: 17, or a variant thereof having at least about 80% sequence identity to the amino acid sequence of SEQ ID NO: 17.

In some embodiments, the antibody specifically recognizing the human TNFR2 comprises a V_{H} comprising the amino acid sequence of SEQ ID NO: 10, or a variant thereof having at least about 80% sequence identity to the amino acid sequence of SEQ ID NO: 10; and a V_{L} comprising the amino acid sequence of SEQ ID NO: 20, or a variant thereof having at least about 80% sequence identity to the amino acid sequence of SEQ ID NO: 20.

In some embodiments, the antibody specifically recognizing the human TNFR2 comprises a V_{H} comprising the amino acid sequence of SEQ ID NO: 11, or a variant thereof having at least about 80% sequence identity to the amino acid sequence of SEQ ID NO: 11; and a V_{L} comprising the amino acid sequence of SEQ ID NO: 20, or a variant thereof having at least about 80% sequence identity to the amino acid sequence of SEQ ID NO: 20.

In some embodiments, the antibody specifically recognizing the human TNFR2 comprises a V_{H} comprising the amino acid sequence of SEQ ID NO: 12, or a variant thereof having at least about 80% sequence identity to the amino acid sequence of SEQ ID NO: 12; and a V_{L} comprising the amino acid sequence of SEQ ID NO: 20, or a variant thereof having at least about 80% sequence identity to the amino acid sequence of SEQ ID NO: 20.

In some embodiments, the antibody specifically recognizing the human TNFR2 comprises a V_{H} comprising the amino acid sequence of SEQ ID NO: 13, or a variant thereof having at least about 80% sequence identity to the amino acid sequence of SEQ ID NO: 13; and a V_{L} comprising the amino acid sequence of SEQ ID NO: 20, or a variant thereof having at least about 80% sequence identity to the amino acid sequence of SEQ ID NO: 20.

In some embodiments, the antibody specifically recognizing the human TNFR2 comprises a V_{H} comprising the amino acid sequence of SEQ ID NO: 14, or a variant thereof having at least about 80% sequence identity to the amino acid sequence of SEQ ID NO: 14; and a V_{L} comprising the amino acid sequence of SEQ ID NO: 20, or a variant thereof having at least about 80% sequence identity to the amino acid sequence of SEQ ID NO: 20.

In some embodiments, the antibody specifically recognizing the human TNFR2 comprises a V_{H} comprising the amino acid sequence of SEQ ID NO: 15, or a variant thereof having at least about 80% sequence identity to the amino acid sequence of SEQ ID NO: 15; and a V_{L} comprising the amino acid sequence of SEQ ID NO: 20, or a variant thereof having at least about 80% sequence identity to the amino acid sequence of SEQ ID NO: 20.

In some embodiments, the antibody specifically recognizing the human TNFR2 comprises a heavy chain comprising the amino acid sequence of SEQ ID NO: 28 or a variant thereof having at least about 80% sequence identity to SEQ ID NO: 28; and a light chain comprising the amino acid sequence of SEQ ID NO: 37 or a variant thereof having at least about 80% sequence identity to SEQ ID NO: 37.

In some embodiments, the antibody specifically recognizing the human TNFR2 comprises a heavy chain comprising the amino acid sequence of SEQ ID NO: 29 or a variant thereof having at least about 80% sequence identity to SEQ ID NO: 29; and a light chain comprising the amino acid sequence of SEQ ID NO: 38 or a variant thereof having at least about 80% sequence identity to SEQ ID NO: 38.

In some embodiments, the antibody specifically recognizing the human TNFR2 comprises a heavy chain comprising the amino acid sequence of SEQ ID NO: 30 or a variant thereof having at least about 80% sequence identity to SEQ ID NO: 30; and a light chain comprising the amino acid sequence of SEQ ID NO: 39 or a variant thereof having at least about 80% sequence identity to SEQ ID NO: 39.

In some embodiments, the antibody specifically recognizing the human TNFR2 comprises a heavy chain comprising the amino acid sequence of SEQ ID NO: 30 or a variant thereof having at least about 80% sequence identity to SEQ ID NO: 30; and a light chain comprising the amino acid sequence of SEQ ID NO: 40 or a variant thereof having at least about 80% sequence identity to SEQ ID NO: 40.

In some embodiments, the antibody specifically recognizing the human TNFR2 comprises a heavy chain comprising the amino acid sequence of SEQ ID NO: 30 or a variant thereof having at least about 80% sequence identity to SEQ ID NO: 30; and a light chain comprising the amino acid sequence of SEQ ID NO: 38 or a variant thereof having at least about 80% sequence identity to SEQ ID NO: 38.

In some embodiments, the antibody specifically recognizing the human TNFR2 comprises a heavy chain comprising the amino acid sequence of SEQ ID NO: 31 or a variant thereof having at least about 80% sequence identity to SEQ ID NO: 31; and a light chain comprising the amino acid sequence of SEQ ID NO: 41 or a variant thereof having at least about 80% sequence identity to SEQ ID NO: 41.

In some embodiments, the antibody specifically recognizing the human TNFR2 comprises a heavy chain comprising the amino acid sequence of SEQ ID NO: 32 or a variant thereof having at least about 80% sequence identity to SEQ ID NO: 32; and a light chain comprising the amino acid sequence of SEQ ID NO: 41 or a variant thereof having at least about 80% sequence identity to SEQ ID NO: 41.

In some embodiments, the antibody specifically recognizing the human TNFR2 comprises a heavy chain comprising the amino acid sequence of SEQ ID NO: 33 or a variant thereof having at least about 80% sequence identity to SEQ ID NO: 33; and a light chain comprising the amino acid sequence of SEQ ID NO: 41 or a variant thereof having at least about 80% sequence identity to SEQ ID NO: 41.

In some embodiments, the antibody specifically recognizing the human TNFR2 comprises a heavy chain comprising the amino acid sequence of SEQ ID NO: 34 or a variant thereof having at least about 80% sequence identity to SEQ ID NO: 34; and a light chain comprising the amino acid sequence of SEQ ID NO: 41 or a variant thereof having at least about 80% sequence identity to SEQ ID NO: 41.

In some embodiments, the antibody specifically recognizing the human TNFR2 comprises a heavy chain comprising the amino acid sequence of SEQ ID NO: 35 or a variant thereof having at least about 80% sequence identity to SEQ ID NO: 35; and a light chain comprising the amino acid sequence of SEQ ID NO: 41 or a variant thereof having at least about 80% sequence identity to SEQ ID NO: 41.

In some embodiments, the antibody specifically recognizing the human TNFR2 comprises a heavy chain comprising the amino acid sequence of SEQ ID NO: 36 or a variant thereof having at least about 80% sequence identity to SEQ ID NO: 36; and a light chain comprising the amino acid sequence of SEQ ID NO: 41 or a variant thereof having at least about 80% sequence identity to SEQ ID NO: 41.

In some embodiments, the antibody specifically recognizes the human PD-L1 described above is selected from the group consisting of avelumab, durvalumab, atezolizumab, KN035, CK-301, ADG-104, BCD-135, garivulimab, CBT-502, HLX-20, IMC-001, tagitanlimab, LAE-005, LP-002, MSB-2311, adebrelimab, sugemalimab and socazolimab.

In some embodiments, the antibody specifically recognizes the human PD-1 described above is selected from the group consisting of cemiplimab, nivolumab, pembrolizumab, sintilimab, camrelizumab, toripalimab, tislelizumab, zimberelimab, prolgolimab, dostarlimab, AMG-404, balstilimab, budigalimab, cetrelimab, ezabenlimab, genolimzumab, LZM-009, nofazinlimab, penpulimab, pimivalimab, pucotenlimab, QL-1604, retifanlimab, rulonilimab, sasanlimab, SCT-I10A, serplulimab, SG-001, spartalizumab, SYSA-1802 and TY-101.

In some embodiments, there is provided isolated nucleic acid molecule(s) that encodes any one of the antibodies recognizing human TNFR2, antibodies recognizing human PD-L1 or PD-1. In some embodiments, there is provided a vector comprising any one of the nucleic acid molecules described above. In some embodiments, there is provided a host cell comprising any one of the antibodies described above, any one of the nucleic acid molecules described above, or any one of the vectors described above. In some embodiments, there is provided a method of producing an anti-TNFR2 antibody, an anti-PD-L1 or anti-PD-1 antibody comprising: a) culturing any one of the host cells described above under conditions effective to express the anti-TNFR2 antibody, the anti-PD-L1 or anti-PD-1 antibody; and b) obtaining the expressed anti-TNFR2 antibody, anti-PD-L1 or anti-PD-1 antibody from the host cell.

Also provided are pharmaceutical compositions, kits and articles of manufacture comprising any one or more of the anti-TNFR2 and/or anti-PD-L1 or anti-PD-1 antibodies, nucleic acids, vectors, and isolated host cells described above.

### BRIEF DESCRIPTION OF THE DRAWINGS

FIGS. 1A-1B show the epitope mapping results of the exemplary humanized antibody SB1901-76 using an alanine scanning assay.

FIG. 2A shows the binding assay results of the exemplary anti-TNFR2 antibodies as analyzed by FACS. FIG. 2B shows the ligand blocking assay results of the exemplary anti-TNFR2 antibodies as analyzed by FACS.

FIG. 3 shows the results of the exemplary humanized anti-TNFR2 antibodies in vitro human primary Treg cell proliferation assay.

FIG. 4A showed the tumor growth curves of monotherapies or in combination with an anti-PD-L1 antibody. The average tumor volume changes in each group were shown in FIG. 4B.

FIG. 4C showed the tumor growth curves of monotherapies or in combination with an anti-PD-1 antibody. The average tumor volume changes in each group were shown in FIG. 4D.

### DETAILED DESCRIPTION OF THE APPLICATION

### Definitions

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., systemic spread of a pathogen) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of infection (such as, for example, host cell lysis or necrosis). The methods of the application contemplate any one or more of these aspects of treatment.

The term “prevent,” and similar words such as “prevented,” “preventing,” “prevention” or “prophylactic” etc., indicate an approach for preventing, inhibiting, or reducing the likelihood of the occurrence or recurrence of, a disease or condition, e.g., a pathogenic infection. It also refers to delaying the occurrence or recurrence of a disease or condition, or delaying the occurrence or recurrence of the symptoms of a disease or condition. As used herein, “prevention” and similar words also includes reducing the intensity, effect, symptoms and/or burden of a disease or condition prior to occurrence or recurrence of the disease or condition. As used herein, “prevention” and similar words also includes reducing the risk and susceptibility to occurrence or recurrence of the disease or condition, e.g., a pathogenic infection.

The term “antibody” herein is used in the broadest sense and encompasses a variety of antibody structures, including, but not limited to, monoclonal antibodies, polyclonal antibodies, monospecific, multispecific antibodies (e.g., bispecific antibodies), full-length antibodies and antigen-binding fragments thereof, so long as they exhibit the desired antigen binding activity. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (al heavy chain), or IgA2 (α2 heavy chain).

The term “antigen-binding fragment” as used herein includes an fragment of antibody including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment also includes a fusion protein comprising the antibody fragment described above. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, the term “specifically binds,” “specifically recognizing,” or “is specific for” refers to measurable and reproducible interactions, such as binding between a target and an antibody, that is determinative of the presence of the target in the presence of a heterogeneous population of molecules, including biological molecules. For example, an antibody that specifically recognizes a target (which can be an epitope) is an antibody that binds to this target with greater affinity, avidity, more readily, and/or with greater duration than its bindings to other targets. In some embodiments, an antibody that specifically recognizes an antigen reacts with one or more antigenic determinants of the antigen with a binding affinity that is at least about 10 times its binding affinity for other targets.

An “isolated” antibody as used herein refers to an antibody that (1) is not associated with proteins found in nature, (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this application (see U.S. Pat. No. 4,816,567; and Morrison et al., Proc. Natl. Acad. Sci. USA, 81:6851-6855 (1984)).

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skilled in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR of this application is one that binds an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” comprises the stretch of residues C-terminal to a CH2 domain in an Fe region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC₅₀ value) than the parent polypeptide or a native sequence IgG Fc. According to some embodiments, the improvement in binding compared to a parent polypeptide is about 3-fold, such as about any of 5, 10, 25, 50, 60, 100, 150, 200, or up to 500-fold, or about 25% to 1000% improvement in binding. The polypeptide variant which “exhibits decreased binding” to an FcR, binds at least one FcR with lower affinity (e.g., higher apparent Kd or higher IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates ADCC in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g., in an animal model etc. In some embodiments, the variant is from about 5-fold to about 100-fold, e.g. from about 25 to about 50-fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an antibody, combination thereof or composition thereof as disclosed herein, is an amount sufficient to carry out a specifically stated purpose. An “effective amount” can be determined empirically and by known methods relating to the stated purpose.

The term “therapeutically effective amount” refers to an amount of an antibody, combination thereof or composition thereof as disclosed herein, effective to “treat” a disease or disorder in an individual. In the case of cancer, the therapeutically effective amount of the antibody, combination thereof or composition thereof as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the antibody, combination thereof or composition thereof as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that extends the survival of a patient. In some embodiments, the therapeutically effective amount is an amount that improves progression free survival of a patient

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S.

### Food and Drug Administration.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat infection of type X means the method is used to treat infection of types other than X.

As used herein and in the appended claims, the singular forms “a,” “or,” and “the” include plural referents unless the context clearly dictates otherwise.

### Anti-TNFR2 Antibodies

### TNFR2

Tumor necrosis factor (TNF) receptor 2 (TNFR2) is a signaling molecule found on the surface of a subset of potent regulatory T cells (Tregs) that can activate the proliferation of these cells through nuclear factor kappa B (NFκB). TNFR2 is also abundantly expressed on the surface of many human tumors (Vanamee É S. et al., TNFR2: A Novel Target for Cancer Immunotherapy. Trends Mol Med. 2017 November; 23(11):1037-1046). TNFR2 is a cell-surface receptor that regulates cell survival and proliferation (Chen, X. et al. (2007) Interaction of TNF with TNF receptor type 2 promotes expansion and function of mouse CD4+CD25+T regulatory cells. J. Immunol. 179, 154-161) and targeting this receptor has recently emerged as a potential next-generation cancer therapeutic approach (Chen, X. and Oppenheim, J. J. (2017) Targeting TNFR2, an immune checkpoint stimulator and oncoprotein, is a promising treatment for cancer. Sci. Signal. 10, eaa12328). Certain human tumor cells can aberrantly express TNFR2 and tumor infiltrates are dominated by highly suppressive TNFR2+ Tregs (Shimizu, J. et al. (1999) Induction of tumor immunity by removing CD25+CD4+ T cells: a common basis between tumor immunity and autoimmunity. J. Immunol. 163, 5211-5218, Ungewickell, A. et al. (2015) Genomic analysis of mycosis fungoides and Sezary syndrome identifies recurrent alterations in TNFR2. Nat. Genet. 47, 1056-1060).

The amino acid sequence of an exemplary extracellular domain (ECD) of human TNFR2 comprises or consists of the amino acid sequence of SEQ ID NO: 25.

### Anti-TNFR2 Antibody

In some embodiments, the anti-TNFR2 antibodies disclosed herein block both TNFα 5 from binding to TNFR2 and TNFR2 signaling. That the anti-TNFR2 antibodies block TNFα from binding to TNFR2 means herein that an antibody molecule that binds to the receptor TNFR2 thus prevents the ligand TNFα from binding to the same receptor. That the anti-TNFR2 antibodies disclosed herein block TNFR2 signaling means that they block TNFR2 mediated cell activation. In some embodiments, the anti-TNFR2 antibodies disclosed herein have a depleting effect on TNFR2 positive cells means that upon administration to a patient, such as a human, such an antibody molecule binds specifically to TNFR2 expressed on the surface of TNFR2 positive cells, and this binding results in depletion of such target cells. As mentioned above, TNFR2 is highly expressed on Tregs found in tumors in various cancer patients, and in such patients, the antibody molecule of the invention will preferentially bind to Tregs and thus result in the depletion of Tregs. Tregs have an inhibiting effect on the proliferation, activation and cytotoxic capacity of other immune cells such as CD8 positive (CD8+) cells, and therefore depletion of Tregs will, at least indirectly, result in increased proliferation, activation and possibly migration of CD8+ cells and thus an increase of the number of intratumoral CD8+ cells.

In one aspect, the present application provides anti-TNFR2 antibodies that specifically bind to human and/or cynomolgus monkey TNFR2. Anti-TNFR2 antibodies include, but are not limited to, humanized antibodies, chimeric antibodies, mouse antibodies, human antibodies, and antibodies comprising the heavy chain and/or light chain CDRs discussed herein. In one aspect, the present application provides isolated antibodies that bind to TNFR2. Contemplated anti-TNFR2 antibodies include, for example, full-length anti-TNFR2 antibodies (e.g., full-length IgG1 or IgG4), anti-TNFR2 scFvs, anti-TNFR2 Fc fusion proteins, multi-specific (such as bispecific) anti-TNFR2 antibodies, anti-TNFR2 immunoconjugates, and the like. In some embodiments, the anti-TNFR2 antibody is a full-length antibody (e.g., full-length IgG1 or IgG4) or antigen-binding fragment thereof, which specifically binds to TNFR2. In some 30 embodiments, the anti-TNFR2 antibody is a Fab, a Fab′, a F(ab)′2, a Fab′-SH, a single-chain Fv (scFv), an Fv fragment, a dAb, a Fd, a nanobody, a diabody, or a linear antibody.

In some embodiments, the anti-TNFR2 antibody described herein specifically binds to a linear epitope within human TNFR2. In some embodiments, the anti-TNFR2 antibody described herein specifically binds to a nonlinear epitope within human TNFR2. In some embodiments, the anti-TNFR2 antibody described herein specifically binds to an epitope on human TNFR2, wherein the epitope comprises Arg99, Lys108, Glu110, Gly111, Arg113, Leu114, and Asp136 of human TNFR2 sequence set forth in SEQ ID NO: 25.

In some embodiments, the characteristics comprise, but are not limited to: (i) inhibiting TNF-α binding to TNFR2; (ii) inhibiting TNFR2 signaling; (iii) cross-reactively binding to human TNFR2 and cynomolgus monkey TNFR2; (iv) lower non-specific binding to dsDNA, insulin or baculovirus particles; (v) inhibiting the Treg cell proliferation (vi) suppressing tumor growth or depletion of tumor cells; (vii) reducing Tregs mediated immune suppression; (viii) converting Tregs into effector T cells; (ix) in vivo pharmacokinetics (PK) profiles; (x) thermal stability (e.g. high Tm or Tagg); (xi) developability; (xii) reduced toxicity or immunogenicity; or (xiii) increased ease of manufacturing.

In some embodiments, according to any of the anti-TNFR2 antibodies described herein, the anti-TNFR2 antibody comprises an antibody heavy chain constant region and an antibody light chain constant region. In some embodiments, the anti-TNFR2 antibody comprises an IgG1 heavy chain constant region. In some embodiments, the anti-TNFR2 antibody comprises an IgG2 heavy chain constant region. In some embodiments, the anti-TNFR2 antibody comprises an IgG3 heavy chain constant region. In some embodiments, the anti-TNFR2 antibody comprises an IgG4 heavy chain constant region. In some embodiments, the heavy chain constant region comprises (including consisting of or consisting essentially of) the amino acid sequence of SEQ ID NO: 21. In some embodiments, the heavy chain constant region comprises (including consisting of or consisting essentially of) the amino acid sequence of SEQ ID NO: 22. In some embodiments, the anti-TNFR2 antibody comprises a kappa light chain constant region. In some embodiments, the light chain constant region comprises (including consisting of or consisting essentially of) the amino acid sequence of SEQ ID NO: 23. In some embodiments, the anti-TNFR2 antibody comprises a lambda light chain constant region. In some embodiments, the light chain constant region comprises (including consisting of or consisting essentially of) the amino acid sequence of SEQ ID NO: 24. In some embodiments, the anti-TNFR2 antibody comprises an antibody heavy chain variable domain and an antibody light chain variable domain.

Exemplary antibody sequences are shown in Table 2-4, wherein the CDR numbering is according to the EU index of Kabat. Those skilled in the art will recognize that many algorithms are known for the prediction of CDR positions and delimitation of antibody heavy chain and light chain variable regions. Anti-TNFR2 specifically recognizing TNFR2 comprising CDRs, V_{H} and/or V_{L} sequences from antibodies described herein, but based on prediction algorithms other than those exemplified in the tables below, are within the scope of this invention. The anti-TNFR2 antibody sequences of our company patent with international application No. PCT/US2022/073523 was incorporated here in this invention.

TABLE 2
Exemplary anti-TNFR2 antibody CDR sequences

| Antibody Name | | | |
|---|---|---|---|
| 51B5 and SB1901-19/25/ | HC-CDR1 | HC-CDR2 | HC-CDR3 |
| 26/27/72/74/76/78/80/82 | DDYID | EIYPGSGNTYYNEKFKG | SQVYGKIAMDH |
| | (SEQ ID NO: 1) | (SEQ ID NO: 2) | (SEQ ID NO: 3) |
| | LC-CDR1 | LC-CDR2 | LC-CDR3 |
| | RASESVDNSGNSFMH | RASNLES | QQSKEDPYT |
| | (SEQ ID NO: 4) | (SEQ ID NO: 5) | (SEQ ID NO: 6) |

TABLE 3
Exemplary VH and VL sequences

| SEQ ID | | |
|---|---|---|
| NO | Description | Sequence |
| 7 | 51B5 V_{H} | QVQLQQSGAELARPGTSVKLSCKASGYTFTDDYIDWVKQRTGQGL |
| | | EWIGEIYPGSGNTYYNEKFKGKATLTADKSSSTAYMQLSSLTSEDS |
| | | AVYFCARSQVYGKIAMDHWGQGTSVTVSS |
| 8 | SB1901-19 V_{H} | QVQLVQSGAEVKKPGASVKVSCKASGYTFTDDYIDWVRQATGQG |
| | | LEWMGEIYPGSGNTYYNEKFKGRATMTRNTSISTAYMELSSLRSED |
| | | TAVYYCARSQVYGKIAMDHWGQGTLVTVSS |
| 9 | SB1901-25 V_{H} | QVQLVQSGAEVKKPGASVKVSCKASGYTFTDDYIDWVRQATGQG |
| | SB1901-26 V_{H} | LEWMGEIYPGSGNTYYNEKFKGRVTLTRNTSISTAYMELSSLRSED |
| | SB1901-27 V_{H} | TAVYYCARSQVYGKIAMDHWGQGTLVTVSS |
| 10 | SB1901-72 V_{H} | QVQLVQSGAEVKKPGASVKVSCKASGYTFTDDYIDWVRQATGQG |
| | | LEWIGEIYPGSGNTYYNEKFKGRVTMTRDKSISTAYMELSSLRSED |
| | | TAVYYCARSQVYGKIAMDHWGQGTLVTVSS |
| 11 | SB1901-76 V_{H} | QVQLVQSGAEVKKPGASVKVSCKASGYTFTDDYIDWVRQATGQG |
| | | LEWIGEIYPGSGNTYYNEKFKGRVTMTRNTGISTAYMELSSLRSED |
| | | TAVYYCARSQVYGKIAMDHWGQGTLVTVSS |
| 12 | SB1901-80 V_{H} | QVQLVQSGAEVKKPGASVKVSCKASGYTFTDDYIDWVRQAPGQG |
| | | LEWIGEIYPGSGNTYYNEKFKGRVTMTRDTSTSTVYMELSSLRSED |
| | | TAVYYCARSQVYGKIAMDHWGQGTLVTVSS |
| 13 | SB1901-74 V_{H} | QVQLVQSGAEVKKPGASVKVSCKASGYTFTDDYIDWVRQATGQG |
| | | LEWIGEIYPGSGNTYYNEKFKGRVTMTRNTAISTAYMELSSLRSED |
| | | TAVYYCARSQVYGKIAMDHWGQGTLVTVSS |
| 14 | SB1901-78 V_{H} | QVQLVQSGAEVKKPGASVKVSCKASGYTFTDDYIDWVRQATGQG |
| | | LEWIGEIYPGSGNTYYNEKFKGRVTMTADKSISTAYMELSSLRSED |
| | | TAVYYCARSQVYGKIAMDHWGQGTLVTVSS |
| 15 | SB1901-82 V_{H} | QVQLVQSGAEVKKPGASVKVSCKASGYTFTDDYIDWVRQAPGQG |
| | | LEWMGEIYPGSGNTYYNEKFKGRVTMTRDTSTSTVYMELSSLRSE |
| | | DTAVYYCARSQVYGKIAMDHWGQGTLVTVSS |
| 16 | 51B5 V_{L} | DIVLTQSPASLAVSLGQRATISCRASESVDNSGNSFMHWYQQIPGQ |
| | | PPKLLIYRASNLESGIPARFSGSGARTDFTLTIDPVGAVDVATYYCQ |
| | | QSKEDPYTFGGGTKLEIE |
| 17 | SB1901-19 V_{L} | DIVMTQSPDSLAVSLGERATINCRASESVDNSGNSFMHWYQQKPG |
| | SB1901-27 V_{L} | QPPKLLIYRASNLESGVPDRESGSGAGTDFTLTISSLQAEDVAVYYC |
| | | QQSKEDPYTFGQGTKVEIK |
| 18 | SB1901-25 V_{L} | DIVMTQSPDSLAVSLGERATINCRASESVDNSGNSFMHWYQQKPG |
| | | QPPKLLIYRASNLESGVPDRFSGSGSGTDFTLTISSLQAEDVAVYYC |
| | | QQSKEDPYTFGQGTKVEIK |
| 19 | SB1901-26 V_{L} | DIVLTQSPDSLAVSLGERATINCRASESVDNSGNSFMHWYQQKPGQ |
| | | PPKLLIYRASNLESGVPDRFSGSGSGTDFTLTISSLQAEDVAVYYCQ |
| | | QSKEDPYTFGQGTKVEIK |
| 20 | SB1901-72 V_{L} | DIVMTQSPDSLAVSLGERATINCRASESVDNSGNSFMHWYQQKPG |
| | SB1901-74 V_{L} | QPPKLLIYRASNLESGVPDRFSGSGSRTDFTLTISSLQAEDVAVYYC |
| | SB1901-76 V_{L} | QQSKEDPYTFGQGTKVEIK |
| | SB1901-78 V_{L} | |
| | SB1901-80 V_{L} | |
| | SB1901-82 V_{L} | |

TABLE 4
Exemplary Sequences

| SEQ ID | | |
|---|---|---|
| NO: | Description | Sequence |
| 21 | IgG1 heavy chain | ASTKGPSVFPLAPSSKSTSGGTAALGCLVKDYFPEPVTVSWNSG |
| | constant region | ALTSGVHTFPAVLQSSGLYSLSSVVTVPSSSLGTQTYICNVNHK |
| | | PSNTKVDKKVEPKSCDKTHTCPPCPAPELLGGPSVFLFPPKPKD |
| | | TLMISRTPEVTCVVVDVSHEDPEVKFNWYVDGVEVHNAKTKP |
| | | REEQYNSTYRVVSVLTVLHQDWLNGKEYKCKVSNKALPAPIE |
| | | KTISKAKGQPREPQVYTLPPSREEMTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 22 | IgG4 heavy chain | ASTKGPSVFPLAPCSRSTSESTAALGCLVKDYFPEPVTVSWNSG |
| | constant region | ALTSGVHTFPAVLQSSGLYSLSSVVTVPSSSLGTKTYTCNVDHK |
| | | PSNTKVDKRVESKYGPPCPSCPAPEFLGGPSVFLFPPKPKDTLMI |
| | | SRTPEVTCVVVDVSQEDPEVQFNWYVDGVEVHNAKTKPREEQ |
| | | FNSTYRVVSVLTVLHQDWLNGKEYKCKVSNKGLPSSIEKTISK |
| | | AKGQPREPQVYTLPPSQEEMTKNQVSLTCLVKGFYPSDIAVEW |
| | | ESNGQPENNYKTTPPVLDSDGSFFLYSRLTVDKSRWQEGNVFS |
| | | CSVMHEALHNHYTQKSLSLSLGK |
| 23 | Light chain constant | RTVAAPSVFIFPPSDEQLKSGTASVVCLLNNFYPREAKVQWKV |
| | region(kappa) | DNALQSGNSQESVTEQDSKDSTYSLSSTLTLSKADYEKHKVYA |
| | | CEVTHQGLSSPVTKSFNRGEC |
| 24 | Light chain constant | GQPKAAPSVTLFPPSSEELQANKATLVCLISDFYPGAVTVAWK |
| | region(lambda) | ADSSPVKAGVETTTPSKQSNNKYAASSYLSLTPEQWKSHRSYS |
| | | CQVTHEGSTVEKTVAPTECS |
| 28 | 51B5 heavy chain | QVQLQQSGAELARPGTSVKLSCKASGYTFTDDYIDWVKQRTG |
| | | QGLEWIGEIYPGSGNTYYNEKFKGKATLTADKSSSTAYMQLSS |
| | | LTSEDSAVYFCARSQVYGKIAMDHWGQGTSVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHTCPPCPAPELLGGPSVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSREEMTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK |
| 29 | SB1901-19 heavy | QVQLVQSGAEVKKPGASVKVSCKASGYTFTDDYIDWVRQATG |
| | chain | QGLEWMGEIYPGSGNTYYNEKFKGRATMTRNTSISTAYMELSS |
| | | LRSEDTAVYYCARSQVYGKIAMDHWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 30 | SB1901-25/ | QVQLVQSGAEVKKPGASVKVSCKASGYTFTDDYIDWVRQATG |
| | SB1901-26/ | QGLEWMGEIYPGSGNTYYNEKFKGRVTLTRNTSISTAYMELSS |
| | SB1901-27 heavy | LRSEDTAVYYCARSQVYGKIAMDHWGQGTLVTVSSASTKGPS |
| | chain | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 31 | SB1901-72 heavy | QVQLVQSGAEVKKPGASVKVSCKASGYTFTDDYIDWVRQATG |
| | chain | QGLEWIGEIYPGSGNTYYNEKFKGRVTMTRDKSISTAYMELSSL |
| | | RSEDTAVYYCARSQVYGKIAMDHWGQGTLVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHTCPPCPAPELLGGPSVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSREEMTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK |
| 32 | SB1901-76 heavy | QVQLVQSGAEVKKPGASVKVSCKASGYTFTDDYIDWVRQATG |
| | chain | QGLEWIGEIYPGSGNTYYNEKFKGRVTMTRNTGISTAYMELSS |
| | | LRSEDTAVYYCARSQVYGKIAMDHWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 33 | SB1901-80 heavy | QVQLVQSGAEVKKPGASVKVSCKASGYTFTDDYIDWVRQAPG |
| | chain | QGLEWIGEIYPGSGNTYYNEKFKGRVTMTRDTSTSTVYMELSS |
| | | LRSEDTAVYYCARSQVYGKIAMDHWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 34 | SB1901-74 heavy | QVQLVQSGAEVKKPGASVKVSCKASGYTFTDDYIDWVRQATG |
| | chain | QGLEWIGEIYPGSGNTYYNEKFKGRVTMTRNTAISTAYMELSS |
| | | LRSEDTAVYYCARSQVYGKIAMDHWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 35 | SB1901-78 heavy | QVQLVQSGAEVKKPGASVKVSCKASGYTFTDDYIDWVRQATG |
| | chain | QGLEWIGEIYPGSGNTYYNEKFKGRVTMTADKSISTAYMELSS |
| | | LRSEDTAVYYCARSQVYGKIAMDHWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 36 | SB1901-82 heavy | QVQLVQSGAEVKKPGASVKVSCKASGYTFTDDYIDWVRQAPG |
| | chain | QGLEWMGEIYPGSGNTYYNEKFKGRVTMTRDTSTSTVYMELS |
| | | SLRSEDTAVYYCARSQVYGKIAMDHWGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 37 | 51B5 light chain | DIVLTQSPASLAVSLGQRATISCRASESVDNSGNSFMHWYQQIP |
| | | GQPPKLLIYRASNLESGIPARFSGSGARTDFTLTIDPVGAVDVAT |
| | | YYCQQSKEDPYTFGGGTKLEIERTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| 38 | SB1901-19/ | DIVMTQSPDSLAVSLGERATINCRASESVDNSGNSFMHWYQQK |
| | SB1901-27 light | PGQPPKLLIYRASNLESGVPDRESGSGAGTDFTLTISSLQAEDVA |
| | chain | VYYCQQSKEDPYTFGQGTKVEIKRTVAAPSVFIFPPSDEQLKSG |
| | | TASVVCLLNNFYPREAKVQWKVDNALQSGNSQESVTEQDSKD |
| | | STYSLSSTLTLSKADYEKHKVYACEVTHQGLSSPVTKSENRGEC |
| 39 | SB1901-25 light | DIVMTQSPDSLAVSLGERATINCRASESVDNSGNSFMHWYQQK |
| | chain | PGQPPKLLIYRASNLESGVPDRFSGSGSGTDFTLTISSLQAEDVA |
| | | VYYCQQSKEDPYTFGQGTKVEIKRTVAAPSVFIFPPSDEQLKSG |
| | | TASVVCLLNNFYPREAKVQWKVDNALQSGNSQESVTEQDSKD |
| | | STYSLSSTLTLSKADYEKHKVYACEVTHQGLSSPVTKSFNRGEC |
| 40 | SB1901-26 light | DIVLTQSPDSLAVSLGERATINCRASESVDNSGNSFMHWYQQK |
| | chain | PGQPPKLLIYRASNLESGVPDRFSGSGSGTDFTLTISSLQAEDVA |
| | | VYYCQQSKEDPYTFGQGTKVEIKRTVAAPSVFIFPPSDEQLKSG |
| | | TASVVCLLNNFYPREAKVQWKVDNALQSGNSQESVTEQDSKD |
| | | STYSLSSTLTLSKADYEKHKVYACEVTHQGLSSPVTKSFNRGEC |
| 41 | SB1901-72/ | DIVMTQSPDSLAVSLGERATINCRASESVDNSGNSFMHWYQQK |
| | SB1901-74/ | PGQPPKLLIYRASNLESGVPDRESGSGSRTDFTLTISSLQAEDVA |
| | SB1901-76/ | VYYCQQSKEDPYTFGQGTKVEIKRTVAAPSVFIFPPSDEQLKSG |
| | SB1901-78/ | TASVVCLLNNFYPREAKVQWKVDNALQSGNSQESVTEQDSKD |
| | SB1901-80/ | STYSLSSTLTLSKADYEKHKVYACEVTHQGLSSPVTKSENRGEC |
| | SB1901-82 light | |
| | chain | |

### PD-L1 or PD-1 Antagonist

The PD-L1 or PD-1 antagonist can be any agent that inhibits the biological activity of PD-L1 or PD-1. As used herein, “PD-L1 or PD-1 biological activity” includes PD-L1 or PD-1 biological activity or both PD-L1 or PD-1 biological activity. As used herein, the term “PD-L1 or PD-1 antagonist” collectively refers to PD-L1 or PD-1 antagonists. The biological activity of PD-L1 or PD-1 may be inhibited in any manner, e.g., by inhibiting the expression of any one or more of PD-L1 or PD-L1 mRNA, PD-L1 or PD-1 protein, or by inhibiting the binding of PD-L1 to PD-1. The biological activity may be inhibited to any degree that realizes a beneficial therapeutic effect. For example, in some embodiments, the biological activity may be completely inhibited (i.e., prevented), while in other embodiments, the biological activity may be partially inhibited (i.e., reduced). As used herein, unless stated otherwise, the terms “PD-L1 or PD-1” refer to PD-L1 or PD-1 respectively, in any form (e.g., mRNA or protein) and from any species (e.g., human or mouse).

In some embodiments of the invention, the PD-L1 or PD-1 antagonist is an agent that inhibits PD-L1 or PD-1 signaling, PD-L1 or PD-1 signaling can be inhibited in any manner. For example, the PD-L1 or PD-1 antagonist may inhibit the activation and/or binding of any one or more of various downstream targets of PD-L1 or PD-1-signaling (e.g., nuclear factor kappa-light-chain-enhancer of activated B cells (NFκB), mitogen-activated protein kinase (MAPK) 3/extracellular signal-regulated kinase (ERK) 1, MAPK8/Jun N-Terminal Protein Kinase (JNK), AKT, and B-catenin). For example, the PD-L1 or PD-1 antagonist may be an agent that binds to PD-L1 or PD-1, thereby reducing or preventing PD-L1 or PD-1 signaling and inhibiting its function. By way of illustration, the PD-L1 or PD-1 antagonist that inhibits PD-L1 or PD-1-signaling can be any of the antibodies or antibody fragments, antisense nucleic acids, or chemical inhibitors (e.g., small molecule or peptide (or polypeptide) inhibitor) described herein.

In an embodiment, the PD-L1 or PD-1 antagonist is an agent that inhibits the binding of PD-L1 to PD-1. In this regard, the PD-L1 or PD-1 antagonist may be any agent that binds to the PD-L1 or PD-1 protein, thereby reducing or preventing the binding of PD-L1 to PD-1 and inhibiting its function, as well as agents that compete with the PD-L1 or PD-1 protein for the native PD-L1 or PD-1 binding site. By way of illustration, the agent that inhibits the binding of PD-L1 or PD-1 to any of its binding sites can be any of the antibodies or antibody fragments, antisense nucleic acids, or chemical inhibitors (e.g., small molecule or peptide inhibitor) described herein.

In an embodiment of the invention, the PD-L1 or PD-1 antagonist is an antibody specifically recognizing human PD-L1 or PD-1. In some embodiments, the antagonist is an antibody fragment that specifically binds to human PD-L1 or PD-1. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments. The antibody can be in monomeric or polymeric form. In some embodiments, the antibody to PD-L1 or PD-1 specifically binds to human PD-L1 or human PD-1. The antibody may be a human antibody, a humanized antibody or a chimeric antibody, Also, the antibody can have any level of affinity or avidity for PD-L1 or PD-1, or a functional domain of PD-L1 or PD-1, e.g., the binding portion of PD-L1 or PD-1.

Anti-PD-L1 or anti-PD-1 antibodies and antibody fragments can be prepared using the PD-L1 or PD-1 proteins disclosed herein and routine techniques. Suitable methods of making antibodies are known in the art. For instance, standard hybridoma methods are described in, e.g., Kohler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, NY (2001)). Alternatively, other methods, such as EBV-hybridoma methods (Haskard and Archer, J. Immunol. Methods, 74(2), 361-67 (1984), and Roder et al, Methods Enzymol, 121, 140-67 (1986)), and bacteriophage vector expression systems (see, e.g., Huse et al, Science, 246, 1275-81 (1989)) are known in the art. Further, methods of producing antibodies in non-human animals are described in, e.g., U.S. Pat. Nos. 5,545,806, 5,569,825, and 5,714,352, U.S. Patent Application Publication No. 2002/0197266 A1, and U.S. Pat. No. 7,338,929)

In some embodiments, examples of antibodies that bind to PD-1, and are useful in the various aspects and embodiments of the present invention, are described in U.S. Pat. Nos. 8,552,154; 8,354,509; 8,168,757; 8,008,449; 7,521,051; 7,488,802; WO2004072286; WO2004056875; and WO2004004771. In some embodiments, the anti-PD-1 antibody is selected from the group consisting of cemiplimab, nivolumab, pembrolizumab, sintilimab, camrelizumab, toripalimab, tislelizumab, zimberelimab, prolgolimab, dostarlimab, AMG-404, balstilimab, budigalimab, cetrelimab, ezabenlimab, genolimzumab, LZM-009, nofazinlimab, penpulimab, pimivalimab, pucotenlimab, QL-1604, retifanlimab, rulonilimab, sasanlimab, SCT-I10A, serplulimab, SG-001, spartalizumab, SYSA-1802 and TY-101.

In some embodiments, examples of antibodies that bind to PD-L1 are disclosed in U.S. Pat. Nos. 9,212,224; 8,779,108; 8,552,154; 8,383,796; 8,217,149; US Patent Publication No. 20110280877; WO2013079174; and WO2013019906. Additional exemplary antibodies that bind to PD-L1 (also referred to as CD274 or B7-H1) and methods for use are disclosed in U.S. Pat. Nos. 8,168,179; 7,943,743; 7,595,048; WO2014055897; WO2013019906; and WO2010077634. Specific anti-human PD-L1 monoclonal antibodies useful as a PD-1 antagonist in the treatment method, medicaments and uses of the present invention include MPDL3280A, BMS-936559, MEDI4736, MSB0010718C. In some embodiments, the anti-PD-L1 antibody is selected from the group consisting of avelumab, durvalumab, atezolizumab, KN035, CK-301, ADG-104, BCD-135, garivulimab, CBT-502, HLX-20, IMC-001, tagitanlimab, LAE-005, LP-002, MSB-2311, adebrelimab, sugemalimab and socazolimab.

### Pharmaceutical Compositions and Combination

Also provided herein are compositions (such as pharmaceutical compositions, also referred to herein as formulations) comprising one or a combination of antibodies, such as a combination of anti-TNFR2 antibodies and anti-PD-L1 or anti-PD-1 antibodies. Nucleic acids encoding the anti-TNFR2 antibodies, anti-PD-L1 or anti-PD-1 antibodies, vectors comprising the nucleic acids encoding any one of the antibodies, or host cells comprising the nucleic acids or vectors described herein. In some embodiments, there is provided a pharmaceutical composition comprising one or a combination of anti-TNFR2 antibodies and anti-PD-L1 or anti-PD-1 antibodies described herein and a pharmaceutically acceptable carrier.

Suitable formulations of the anti-TNFR2 antibodies, anti-PD-L1 or anti-PD-1 antibodies are obtained by mixing an anti-TNFR2 antibody, anti-PD-L1 or anti-PD-1 antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-TNFR2 antibodies of this application into cells.

The formulation herein may also contain one or more active compounds in addition to the anti-TNFR2 antibody, anti-PD-L1 or anti-PD-1 antibody as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-TNFR2 antibody, anti-PD-L1 or anti-PD-1 antibody. Such molecules are suitably present in combination in amounts that are effective for the purpose intended. The effective amount of such other agents depends on the amount of anti-TNFR2 antibody, anti-PD-L1 or anti-PD-1 antibody present in the formulation, the type of disease or disorder or treatment, and other factors discussed above. These are generally used in the same dosages and with administration routes as described herein or about from 1 to 99% of the heretofore employed dosages.

The anti-TNFR2 antibodies, anti-PD-L1 or anti-PD-1 antibodies (e.g., full-length antiTNFR2 antibodies, anti-PD-L1 or anti-PD-1 antibodies) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared.

Sustained-release preparations of the anti-TNFR2 antibodies, anti-PD-L1 or anti-PD-1 antibodies (e.g., full-length anti-TNFR2 antibodies, anti-PD-L1 or anti-PD-1 antibodies) can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D (−)-3-hydroxybutyric acid. While polymers such as ethylene-vinyl acetate and lactic acid-glycolic acid enable release of molecules for over 100 days, certain hydro gels release proteins for shorter time periods. When encapsulated antibody remain in the body for a long time, they can denature or aggregate as a result of exposure to moisture at 37° C., resulting in a loss of biological activity and possible changes in immunogenicity. Rational strategies can be devised for stabilization of anti-TNFR2 antibodies, anti-PD-L1 or anti-PD-1 antibodies depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-TNFR2 antibody, anti-PD-L1 or anti-PD-1 antibody (such as a full-length anti-TNFR2 antibody, anti-PD-L1 or anti-PD-1 antibody) is formulated in a buffer comprising a citrate, NaCl, acetate, succinate, glycine, polysorbate 80 (Tween 80), or any combination of the foregoing. In some embodiments, the anti-TNFR2 antibody, anti-PD-L1 or anti-PD-1 antibody is formulated in a buffer comprising about 100 mM to about 150 mM glycine. In some embodiments, the anti-TNFR2 antibody, anti-PD-L1 or anti-PD-1 antibody is formulated in a buffer comprising NaCl. In some embodiments, the anti-TNFR2 antibody, anti-PD-L1 or anti-PD-1 antibody is formulated in a buffer comprising acetate. In some embodiments, the anti-TNFR2 antibody, anti-PD-L1 or anti-PD-1 antibody is formulated in a buffer comprising succinate. In some embodiments, the anti-TNFR2 antibody, anti-PD-L1 or anti-PD-1 antibody is formulated in a buffer comprising polysorbate 80. In some embodiments, the anti-TNFR2 antibody, anti-PD-L1 or anti-PD-1 antibody is formulated in a buffer having a pH between about 5.1 and 5.6.

The formulations to be used for in vivo administration must be sterile. This is readily accomplished by, e.g., filtration through sterile filtration membranes.

In one aspect, the present application provides a pharmaceutical composition comprising an antibody specifically recognizing human TNFR2 and a PD-L1 or PD-1 antagonist.

In one aspect, the present application provides a pharmaceutical composition comprising an antibody specifically recognizing human TNFR2 and an antibody specifically recognizing PD-L1 or PD-1.

In another aspect, the present application provides a combination of an antibody specifically recognizing human TNFR2 and a PD-L1 or PD-1 antagonist.

In another aspect, the present application provides a combination of an antibody specifically recognizing human TNFR2 and an antibody specifically recognizing human PD-L1 or PD-1.

### Combination Therapy

As used herein, the terms “cancer”, “neoplasm” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors”. Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

These diseases include, but are not limited to, non-small cell lung cancer, adrenal gland cancer, bladder cancer, brain cancer, pancreatic adenocarcinoma, breast cancer, colorectal cancer, melanoma, gastroesophageal junction adenocarcinoma, esophageal cancer, esophageal adenocarcinoma, gall bladder cancer, gastric cancer, cervical cancer, gastric adenocarcinoma, head and neck cancer, heart cancer, hepatocellular carcinoma, kidney cancer, liver cancer, mesothelioma, ovarian cancer, pancreatic cancer, prostate cancer, prostate adenocarcinoma, spleen cancer, small cell lung cancer, testicular cancer, thyroid cancer, uterine cancer, and infectious diseases, including, but not limited to Human Papilloma Virus (HPV), Human Immunodeficiency Virus (HIV), Herpes Simplex Virus (HSV), Varicella Zoster Virus (VSV), Cytomegalovirus (CMV), Epstein Barr Virus (EBV), E. coli, Salmonella, Shigella, Staphylococcus aureus, Coliform Bacteria, Chlamydia, Mycobacterium Tuberculosis, Streptococcus, Pneumococcus, Pseudomonas, Campylobacter, Salmonella, Aspergillus Fumigatus, Aspergillus flavus, Cryptococcus Neoformans, and Histoplasma Capsulatum.

In some embodiments, the cancer is breast cancer, colon cancer, cervical cancer, renal cancer, liver cancer (e.g., hepatocellular carcinoma), lung cancer (e.g., NSCLC), ovarian cancer, melanoma, skin cancer (e.g., squamous cell carcinoma or basal cell carcinoma), lymphoma, or leukemia. In certain embodiments, the cancer is melanoma.

By “combination”, “in combination with” or “combined with”, it is not intended to imply that the therapy or the therapeutic agents must be administered at the same time and/or formulated for delivery together (e.g., in the same composition), although these methods and compositions are within the scope described herein. For example, the anti-TNFR2 antibody and the anti-PD-L1 or anti-PD-1 antibody can be administered concurrently or sequentially with (such as prior to, or subsequent to) each other. The agents in the combination can be administered in any order. In general, each agent will be administered at a dose and/or on a time schedule determined for that agent. As will further be appreciated that the therapeutic agents utilized in combination may be administered together in a single composition or administered separately in different compositions. In general, it is expected that therapeutic agents utilized in combination can be utilized at levels that do not exceed the levels at which they are utilized individually. In some embodiments, the levels utilized in combination can be lower than those utilized individually.

In some embodiments, for the treatment of cancer or infectious disease, the antibody specifically recognizing human TNFR2 may be administered in combination with one or more anti-cancer agents, such as the immune checkpoint inhibitor, chemotherapeutic agent, growth inhibitory agent, anti-angiogenesis agent or anti-neoplastic composition.

In some embodiments, the antibody specifically recognizing human TNFR2 is administered with a second therapeutic agent such as an immune checkpoint inhibitor (e.g., an inhibitor of the PD-1 or PD-L1 pathway, anti-PD-L1 antibody or anti-PD-1 antibody), to a subject having a disease in which the stimulation of the immune system would be beneficial, e.g., cancer or infectious diseases.

In one aspect, there is provided a method of treating cancer or infectious disease in an individual in need thereof comprising administering to the individual a composition comprising an antibody specifically recognizing human TNFR2 and an antibody specifically recognizing human PD-L1 or PD-1.

In some embodiments, the pharmaceutical composition comprising an antibody specifically recognizing human TNFR2 and an antibody specifically recognizing human PD-L1 or PD-1 for use in the treatment of cancer or infectious disease in an individual in need thereof is provided.

In some embodiments, the use of the pharmaceutical composition comprising an antibody specifically recognizing human TNFR2 and an antibody specifically recognizing human PD-L1 or PD-1 in the manufacture of a medicament for the treatment of cancer or infectious disease is provided.

In another aspect, there is provided a method of treating cancer or infectious disease in an individual in need thereof comprising administering to the individual a combination of an antibody specifically recognizing human TNFR2 and an antibody specifically recognizing human PD-L1 or PD-1.

In some embodiments, the combination of an antibody specifically recognizing human TNFR2 and an antibody specifically recognizing human PD-L1 or PD-1 for use in the treatment of cancer or infectious disease in an individual in need thereof is provided.

In some embodiments, the use of the combination of an antibody specifically recognizing human TNFR2 and an antibody specifically recognizing human PD-L1 or PD-1 in the manufacture of a medicament for the treatment of cancer or infectious disease is provided.

In another aspect, there is provided a method of treating cancer or infectious disease in an individual in need thereof, comprising administering to the individual an antibody specifically recognizing human TNFR2 and an antibody specifically recognizing human PD-L1 or human PD-1.

In some embodiments, the antibody specifically recognizing human TNFR2 and the antibody specifically recognizing human PD-L1 or PD-1 for use in the treatment of cancer or infectious disease in an individual in need thereof is provided.

In some embodiments, the use of an antibody specifically recognizing human TNFR2 and an antibody specifically recognizing human PD-L1 or PD-1 in the manufacture of a medicament for the treatment of cancer or infectious disease is also provided. In another embodiments, the use of an antibody specifically recognizing human TNFR2 in the manufacture of a medicament for the combined treatment of cancer or infectious disease with an antibody specifically recognizing human PD-L1 or PD-1, is also provided.

### Method of Administration

In some embodiment, the antibody specifically recognizing human TNFR2 and the antibody specifically recognizing human PD-L1 or PD-1 can be administrated sequentially or concurrently.

In some embodiments, the administration of the antibody specifically recognizing human PD-L1 or PD-1 is followed by the administration of the antibody specifically recognizing human TNFR2. In other embodiments, the administration of the antibody specifically recognizing human TNFR2 is followed by the administration of the antibody specifically recognizing human PD-L1 or PD-1.

In some embodiments, the antibody specifically recognizing human PD-L1 or human PD-1 is administered concurrently with an antibody specifically recognizing human TNFR2.

In some embodiments, the antibody specifically recognizing human PD-L1 or PD-1 is administered during the period of treatment with an antibody specifically recognizing human TNFR2.

In some embodiments, the antibody specifically recognizing human TNFR2 is administered during the period of treatment with an antibody specifically recognizing human PD-L1 or PD-1.

In some embodiments, the pharmaceutical composition or the combination of an antibody specifically recognizing human TNFR2 and an antibody specifically recognizing human PD-L1 or PD-1 described herein is administered to the individual in need thereof in a therapeutically effective amount. In some embodiments, the antibody specifically recognizing human TNFR2 and the antibody specifically recognizing human PD-L1 or PD-1 described herein is administered to the individual in need thereof in a therapeutically effective amount.

In some embodiments, wherein the antibody specifically recognizing the human TNFR2 of the application binds to an epitope on human TNFR2, wherein the epitope comprises one, two, three, four, five, six, or seven amino acid residues selected from the group consisting of Arg99, Lys108, Glu110, Gly111, Arg113, Leu114, and Asp136 of human TNFR2 sequence set forth in SEQ ID NO: 25.

In some embodiments, the antibody specifically recognizing the human TNFR2 comprises a V_{H} comprising an HC-CDR1 comprising the amino acid sequence SEQ ID NO: 1, or a variant thereof comprising up to about 3 amino acid substitutions; an HC-CDR2 comprising the amino acid sequence of SEQ ID NO: 2, or a variant thereof comprising up to about 3 amino acid substitutions; and an HC-CDR3 comprising the amino acid sequence of SEQ ID NO: 3, or a variant thereof comprising up to about 3 amino acid substitutions; and a V_{L} comprising: an LC-CDR1 comprising the amino acid sequence of SEQ ID NO: 4, or a variant thereof comprising up to about 3 amino acid substitutions; an LC-CDR2 comprising the amino acid sequence of SEQ ID NO: 5, or a variant thereof comprising up to about 3 amino acid substitutions; and an LC-CDR3 comprising the amino acid sequence of SEQ ID NO: 6, or a variant thereof comprising up to about 3 amino acid substitutions.

In some embodiments, the antibody specifically recognizing the human TNFR2 comprises a V_{H} comprising an HC-CDR1, an HC-CDR2, and an HC-CDR3 of a V_{H} comprising the amino acid sequence of any one of SEQ ID NOs: 7-15; and a V_{L} comprising a LC-CDR1, a LC-CDR2, and a LC-CDR3 of a V_{L} comprising the amino acid sequence of any one of SEQ ID NOs: 16-20.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 7; and a V_{L} comprising a LC-CDR1, a LC-CDR2, and a LC-CDR3 of a V_{L} comprising the amino acid sequence of SEQ ID NOs: 16.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 8; and a V_{L} comprising a LC-CDR1, a LC-CDR2, and a LC-CDR3 of a V_{L} comprising the amino acid sequence of SEQ ID NOs: 17.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 9; and a V_{L} comprising a LC-CDR1, a LC-CDR2, and a LC-CDR3 of a V_{L} comprising the amino acid sequence of SEQ ID NOs: 18.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 9; and a V_{L} comprising a LC-CDR1, a LC-CDR2, and a LC-CDR3 of a V_{L} comprising the amino acid sequence of SEQ ID NOs: 19.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 9; and a V_{L} comprising a LC-CDR1, a LC-CDR2, and a LC-CDR3 of a V_{L} comprising the amino acid sequence of SEQ ID NOs: 17.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 10; and a V_{L} comprising a LC-CDR1, a LC-CDR2, and a LC-CDR3 of a V_{L} comprising the amino acid sequence of SEQ ID NOs: 20.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 11; and a V_{L} comprising a LC-CDR1, a LC-CDR2, and a LC-CDR3 of a V_{L} comprising the amino acid sequence of SEQ ID NOs: 20.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 12; and a V_{L} comprising a LC-CDR1, a LC-CDR2, and a LC-CDR3 of a V_{L} comprising the amino acid sequence of SEQ ID NOs: 20.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 13; and a V_{L} comprising a LC-CDR1, a LC-CDR2, and a LC-CDR3 of a V_{L} comprising the amino acid sequence of SEQ ID NOs: 20.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 14; and a V_{L} comprising a LC-CDR1, a LC-CDR2, and a LC-CDR3 of a V_{L} comprising the amino acid sequence of SEQ ID NOs: 20.

In some embodiments, the antibody specifically recognizing the human TNFR2 comprises a V_{H} comprising an HC-CDR1, an HC-CDR2, and an HC-CDR3 of a V_{H} comprising the amino acid sequence of SEQ ID NOs: 15; and a V_{L} comprising a LC-CDR1, a LC-CDR2, and a LC-CDR3 of a V_{L} comprising the amino acid sequence of SEQ ID NOs: 20.

In some embodiments, the antibody specifically recognizing the human TNFR2 comprises a V_{H} comprising the amino acid sequence of any one of SEQ ID NOs: 7-15, or a variant thereof having at least about 80% (such as at least about any one of 80%, 85%, 90%, 95%, 96%, 97%, 98%, or 99%) sequence identity to the amino acid sequence of any one of SEQ ID NOs: 7-15; and a V_{L} comprising the amino acid sequence of any one of SEQ ID NOs: 16-20, or a variant thereof having at least about 80% (such as at least about any one of 80%, 85%, 90%, 95%, 96%, 97%, 98%, or 99%) sequence identity to the amino acid sequence of any one of SEQ ID NOs: 16-20.

In some embodiments, the antibody specifically recognizing the human TNFR2 comprises a V_{H} comprising the amino acid sequence of SEQ ID NO: 7, or a variant thereof having at least about 80% (such as at least about any one of 80%, 85%, 90%, 95%, 96%, 97%, 98%, or 99%) sequence identity to the amino acid sequence of SEQ ID NO: 7; and a V_{L} comprising the amino acid sequence of SEQ ID NO: 16, or a variant thereof having at least about 80% (such as at least about any one of 80%, 85%, 90%, 95%, 96%, 97%, 98%, or 99%) sequence identity to the amino acid sequence of SEQ ID NO: 16.

In some embodiments, the antibody specifically recognizing the human TNFR2 comprises a V_{H} comprising the amino acid sequence of SEQ ID NO: 8, or a variant thereof having at least about 80% (such as at least about any one of 80%, 85%, 90%, 95%, 96%, 97%, 98%, or 99%) sequence identity to the amino acid sequence of SEQ ID NO: 8; and a V_{L} comprising the amino acid sequence of SEQ ID NO: 17, or a variant thereof having at least about 80% (such as at least about any one of 80%, 85%, 90%, 95%, 96%, 97%, 98%, or 99%) sequence identity to the amino acid sequence of SEQ ID NO: 17.

In some embodiments, the antibody specifically recognizing the human TNFR2 comprises a V_{H} comprising the amino acid sequence of SEQ ID NO: 9, or a variant thereof having at least about 80% (such as at least about any one of 80%, 85%, 90%, 95%, 96%, 97%, 98%, or 99%) sequence identity to the amino acid sequence of SEQ ID NO: 9; and a V_{L} comprising the amino acid sequence of SEQ ID NO: 18, or a variant thereof having at least about 80% (such as at least about any one of 80%, 85%, 90%, 95%, 96%, 97%, 98%, or 99%) sequence identity to the amino acid sequence of SEQ ID NO: 18.

In some embodiments, the antibody specifically recognizing the human TNFR2 comprises a V_{H} comprising the amino acid sequence of SEQ ID NO: 9, or a variant thereof having at least about 80% (such as at least about any one of 80%, 85%, 90%, 95%, 96%, 97%, 98%, or 99%) sequence identity to the amino acid sequence of SEQ ID NO: 9; and a V_{L} comprising the amino acid sequence of SEQ ID NO: 19, or a variant thereof having at least about 80% (such as at least about any one of 80%, 85%, 90%, 95%, 96%, 97%, 98%, or 99%) sequence identity to the amino acid sequence of SEQ ID NO: 19.

In some embodiments, the antibody specifically recognizing the human TNFR2 comprises a V_{H} comprising the amino acid sequence of SEQ ID NO: 9, or a variant thereof having at least about 80% (such as at least about any one of 80%, 85%, 90%, 95%, 96%, 97%, 98%, or 99%) sequence identity to the amino acid sequence of SEQ ID NO: 9; and a V_{L} comprising the amino acid sequence of SEQ ID NO: 17, or a variant thereof having at least about 80% (such as at least about any one of 80%, 85%, 90%, 95%, 96%, 97%, 98%, or 99%) sequence identity to the amino acid sequence of SEQ ID NO: 17.

In some embodiments, the antibody specifically recognizing the human TNFR2 comprises a V_{H} comprising the amino acid sequence of SEQ ID NO: 10, or a variant thereof having at least about 80% (such as at least about any one of 80%, 85%, 90%, 95%, 96%, 97%, 98%, or 99%) sequence identity to the amino acid sequence of SEQ ID NO: 10; and a V_{L} comprising the amino acid sequence of SEQ ID NO: 20, or a variant thereof having at least about 80% (such as at least about any one of 80%, 85%, 90%, 95%, 96%, 97%, 98%, or 99%) sequence identity to the amino acid sequence of SEQ ID NO: 20.

In some embodiments, the antibody specifically recognizing the human TNFR2 comprises a V_{H} comprising the amino acid sequence of SEQ ID NO: 11, or a variant thereof having at least about 80% (such as at least about any one of 80%, 85%, 90%, 95%, 96%, 97%, 98%, or 99%) sequence identity to the amino acid sequence of SEQ ID NO: 11; and a V_{L} comprising the amino acid sequence of SEQ ID NO: 20, or a variant thereof having at least about 80% (such as at least about any one of 80%, 85%, 90%, 95%, 96%, 97%, 98%, or 99%) sequence identity to the amino acid sequence of SEQ ID NO: 20.

In some embodiments, the antibody specifically recognizing the human TNFR2 comprises a V_{H} comprising the amino acid sequence of SEQ ID NO: 12, or a variant thereof having at least about 80% (such as at least about any one of 80%, 85%, 90%, 95%, 96%, 97%, 98%, or 99%) sequence identity to the amino acid sequence of SEQ ID NO: 12; and a V_{L} comprising the amino acid sequence of SEQ ID NO: 20, or a variant thereof having at least about 80% (such as at least about any one of 80%, 85%, 90%, 95%, 96%, 97%, 98%, or 99%) sequence identity to the amino acid sequence of SEQ ID NO: 20.

In some embodiments, the antibody specifically recognizing the human TNFR2 comprises a V_{H} comprising the amino acid sequence of SEQ ID NO: 13, or a variant thereof having at least about 80% (such as at least about any one of 80%, 85%, 90%, 95%, 96%, 97%, 98%, or 99%) sequence identity to the amino acid sequence of SEQ ID NO: 13; and a V_{L} comprising the amino acid sequence of SEQ ID NO: 20, or a variant thereof having at least about 80% (such as at least about any one of 80%, 85%, 90%, 95%, 96%, 97%, 98%, or 99%) sequence identity to the amino acid sequence of SEQ ID NO: 20.

In some embodiments, the antibody specifically recognizing the human TNFR2 comprises a V_{H} comprising the amino acid sequence of SEQ ID NO: 14, or a variant thereof having at least about 80% (such as at least about any one of 80%, 85%, 90%, 95%, 96%, 97%, 98%, or 99%) sequence identity to the amino acid sequence of SEQ ID NO: 14; and a V_{L} comprising the amino acid sequence of SEQ ID NO: 20, or a variant thereof having at least about 80% (such as at least about any one of 80%, 85%, 90%, 95%, 96%, 97%, 98%, or 99%) sequence identity to the amino acid sequence of SEQ ID NO: 20.

In some embodiments, the antibody specifically recognizing the human TNFR2 comprises a V_{H} comprising the amino acid sequence of SEQ ID NO: 15, or a variant thereof having at least about 80% (such as at least about any one of 80%, 85%, 90%, 95%, 96%, 97%, 98%, or 99%) sequence identity to the amino acid sequence of SEQ ID NO: 15; and a V_{L} comprising the amino acid sequence of SEQ ID NO: 20, or a variant thereof having at least about 80% (such as at least about any one of 80%, 85%, 90%, 95%, 96%, 97%, 98%, or 99%) sequence identity to the amino acid sequence of SEQ ID NO: 20.

In some embodiments, the antibody specifically recognizing the human TNFR2 comprises a heavy chain comprising the amino acid sequence of SEQ ID NO: 28, or a variant thereof having at least about 80% (such as at least about any one of 80%, 85%, 90%, 95%, 96%, 97%, 98%, or 99%) sequence identity to the amino acid sequence of SEQ ID NO: 28; and a light chain comprising the amino acid sequence of SEQ ID NO: 37, or a variant thereof having at least about 80% (such as at least about any one of 80%, 85%, 90%, 95%, 96%, 97%, 98%, or 99%) sequence identity to the amino acid sequence of SEQ ID NO: 37.

In some embodiments, the antibody specifically recognizing the human TNFR2 comprises a heavy chain comprising the amino acid sequence of SEQ ID NO: 29, or a variant thereof having at least about 80% (such as at least about any one of 80%, 85%, 90%, 95%, 96%, 97%, 98%, or 99%) sequence identity to the amino acid sequence of SEQ ID NO: 29; and a light chain comprising the amino acid sequence of SEQ ID NO: 38, or a variant thereof having at least about 80% (such as at least about any one of 80%, 85%, 90%, 95%, 96%, 97%, 98%, or 99%) sequence identity to the amino acid sequence of SEQ ID NO: 38.

In some embodiments, the antibody specifically recognizing the human TNFR2 comprises a heavy chain comprising the amino acid sequence of SEQ ID NO: 30, or a variant thereof having at least about 80% (such as at least about any one of 80%, 85%, 90%, 95%, 96%, 97%, 98%, or 99%) sequence identity to the amino acid sequence of SEQ ID NO: 30; and a light chain comprising the amino acid sequence of SEQ ID NO: 39, or a variant thereof having at least about 80% (such as at least about any one of 80%, 85%, 90%, 95%, 96%, 97%, 98%, or 99%) sequence identity to the amino acid sequence of SEQ ID NO: 39.

In some embodiments, the antibody specifically recognizing the human TNFR2 comprises a heavy chain comprising the amino acid sequence of SEQ ID NO: 30, or a variant 10 thereof having at least about 80% (such as at least about any one of 80%, 85%, 90%, 95%, 96%, 97%, 98%, or 99%) sequence identity to the amino acid sequence of SEQ ID NO: 30; and a light chain comprising the amino acid sequence of SEQ ID NO: 40, or a variant thereof having at least about 80% (such as at least about any one of 80%, 85%, 90%, 95%, 96%, 97%, 98%, or 99%) sequence identity to the amino acid sequence of SEQ ID NO: 40.

In some embodiments, the antibody specifically recognizing the human TNFR2 comprises a heavy chain comprising the amino acid sequence of SEQ ID NO: 30, or a variant thereof having at least about 80% (such as at least about any one of 80%, 85%, 90%, 95%, 96%, 97%, 98%, or 99%) sequence identity to the amino acid sequence of SEQ ID NO: 30; and a light chain comprising the amino acid sequence of SEQ ID NO: 38, or a variant thereof having at least about 80% (such as at least about any one of 80%, 85%, 90%, 95%, 96%, 97%, 98%, or 99%) sequence identity to the amino acid sequence of SEQ ID NO: 38.

In some embodiments, the antibody specifically recognizing the human TNFR2 comprises a heavy chain comprising the amino acid sequence of SEQ ID NO: 31, or a variant thereof having at least about 80% (such as at least about any one of 80%, 85%, 90%, 95%, 96%, 97%, 98%, or 99%) sequence identity to the amino acid sequence of SEQ ID NO: 31; and a light chain comprising the amino acid sequence of SEQ ID NO: 41, or a variant thereof having at least about 80% (such as at least about any one of 80%, 85%, 90%, 95%, 96%, 97%, 98%, or 99%) sequence identity to the amino acid sequence of SEQ ID NO: 41.

In some embodiments, the antibody specifically recognizing the human TNFR2 comprises a heavy chain comprising the amino acid sequence of SEQ ID NO: 32, or a variant thereof having at least about 80% (such as at least about any one of 80%, 85%, 90%, 95%, 96%, 97%, 98%, or 99%) sequence identity to the amino acid sequence of SEQ ID NO: 32; and a light chain comprising the amino acid sequence of SEQ ID NO: 41, or a variant thereof having at least about 80% (such as at least about any one of 80%, 85%, 90%, 95%, 96%, 97%, 98%, or 99%) sequence identity to the amino acid sequence of SEQ ID NO: 41.

In some embodiments, the antibody specifically recognizing the human TNFR2 comprises a heavy chain comprising the amino acid sequence of SEQ ID NO: 33, or a variant thereof having at least about 80% (such as at least about any one of 80%, 85%, 90%, 95%, 96%, 97%, 98%, or 99%) sequence identity to the amino acid sequence of SEQ ID NO: 33; and a light chain comprising the amino acid sequence of SEQ ID NO: 41, or a variant thereof having at least about 80% (such as at least about any one of 80%, 85%, 90%, 95%, 96%, 97%, 98%, or 99%) sequence identity to the amino acid sequence of SEQ ID NO: 41.

In some embodiments, the antibody specifically recognizing the human TNFR2 comprises a heavy chain comprising the amino acid sequence of SEQ ID NO: 34, or a variant thereof having at least about 80% (such as at least about any one of 80%, 85%, 90%, 95%, 96%, 97%, 98%, or 99%) sequence identity to the amino acid sequence of SEQ ID NO: 34; and a light chain comprising the amino acid sequence of SEQ ID NO: 41, or a variant thereof having at least about 80% (such as at least about any one of 80%, 85%, 90%, 95%, 96%, 97%, 98%, or 99%) sequence identity to the amino acid sequence of SEQ ID NO: 41.

In some embodiments, the antibody specifically recognizing the human TNFR2 comprises a heavy chain comprising the amino acid sequence of SEQ ID NO: 35, or a variant thereof having at least about 80% (such as at least about any one of 80%, 85%, 90%, 95%, 96%, 97%, 98%, or 99%) sequence identity to the amino acid sequence of SEQ ID NO: 35; and a light chain comprising the amino acid sequence of SEQ ID NO: 41, or a variant thereof having at least about 80% (such as at least about any one of 80%, 85%, 90%, 95%, 96%, 97%, 98%, or 99%) sequence identity to the amino acid sequence of SEQ ID NO: 41.

In some embodiments, the antibody specifically recognizing the human TNFR2 comprises a heavy chain comprising the amino acid sequence of SEQ ID NO: 36, or a variant thereof having at least about 80% (such as at least about any one of 80%, 85%, 90%, 95%, 96%, 97%, 98%, or 99%) sequence identity to the amino acid sequence of SEQ ID NO: 36; and a light chain comprising the amino acid sequence of SEQ ID NO: 41, or a variant thereof having at least about 80% (such as at least about any one of 80%, 85%, 90%, 95%, 96%, 97%, 98%, or 99%) sequence identity to the amino acid sequence of SEQ ID NO: 41.

In some embodiments, wherein the antibody specifically recognizing the human PD-L1 is selected from the group consisting of avelumab, durvalumab, atezolizumab, KN035, CK-301, ADG-104, BCD-135, garivulimab, CBT-502, HLX-20, IMC-001, tagitanlimab, LAE-005, LP-002, MSB-2311, adebrelimab, sugemalimab and socazolimab.

In some embodiments, wherein the antibody specifically recognizing the human PD-1 is selected from the group consisting of cemiplimab, nivolumab, pembrolizumab, sintilimab, camrelizumab, toripalimab, tislelizumab, zimberelimab, prolgolimab, dostarlimab, AMG-404, balstilimab, budigalimab, cetrelimab, ezabenlimab, genolimzumab, LZM-009, nofazinlimab, penpulimab, pimivalimab, pucotenlimab, QL-1604, retifanlimab, rulonilimab, sasanlimab, SCT-I10A, serplulimab, SG-001, spartalizumab, SYSA-1802 and TY-101.

In some embodiments, the combination of an antibody specifically recognizing human TNFR2 with an antibody specifically recognizing human PD-L1 or PD-1 is more efficacious than the same molar mass amount of an antibody specifically recognizing human TNFR2 or the same molar mass amount of an antibody specifically recognizing human PD-L1 or PD-1 separately. In some embodiments, the combination of an antibody specifically recognizing human TNFR2 with an antibody specifically recognizing human PD-L1 or PD-1 enhances 15 antitumor effect by about any one of 10%, 20%, 30%, 40%, 50%, 60%, 70%, 80%, 90%, 100%, 2-fold, 5-fold, 10-fold, 20-fold, 50-fold, or 100-fold more compared to the same molar mass amount of an antibody specifically recognizing human TNFR2 or the same molar mass amount of an antibody specifically recognizing human PD-L1 or PD-1 separately.

### Binding Affinity

Binding affinity can be indicated by Kd, Koff, Kon, or Ka. The term “Koff”, as used herein, is intended to refer to the off-rate constant for dissociation of an antigen-binding domain from the antigen-binding domain/antigen complex, as determined from a kinetic selection set up. The term “Kon”, as used herein, is intended to refer to the on-rate constant for association of an antibody to the antigen to form the antigen-binding domain/antigen complex. The term dissociation constant “Kd”, as used herein, refers to the dissociation constant of a particular antibody-antigen interaction, and describes the concentration of antigen required to occupy one half of all of the antigen-binding domains present in a solution of antibody molecules at equilibrium, and is equal to Koff/Kon. The measurement of Kd presupposes that all binding agents are in solution. In the case where the antigen-binding domain is tethered to a cell wall, e.g., in a yeast expression system, the corresponding equilibrium rate constant is expressed as EC50, which gives a good approximation of Kd. The affinity constant, Ka, is the inverse of the dissociation constant, Kd.

The dissociation constant (Kd) is used as an indicator showing affinity of antigen-binding domain moieties to antigens. For example, easy analysis is possible by the Scatchard method using antibodies marked with a variety of marker agents, as well as by using Biacore (made by Amersham Biosciences), analysis of biomolecular interactions by surface plasmon resonance, according to the user's manual and attached kit. The Kd value that can be derived using these methods is expressed in units of M. An antibody that specifically binds to a target may have a Kd of, for example, ≤10⁻⁷ M, ≤10⁻⁸ M, ≤10⁻⁹ M, ≤10⁻¹⁰ M, ≤10⁻¹¹ M, ≤10⁻¹² M, or ≤10⁻¹³ M.

Binding specificity of the antibody can be determined experimentally by methods known in the art. Such methods comprise, but are not limited to, Western blots, ELISA-, RIA-, ECL-, IRMA-, EIA-, BIAcore-tests and peptide scans.

### Nucleic Acids

Nucleic acid molecules encoding anti-TNFR2 antibodies, anti-PD-L1 or anti-PD-1 antibodies are also contemplated. In some embodiments, there is provided a nucleic acid (or a set of nucleic acids) encoding a full-length anti-TNFR2 antibody, anti-PD-L1 or anti-PD-1 antibody, including any of the full-length anti-TNFR2 or anti-PD-L1 or anti-PD-1 antibodies described herein. In some embodiments, the nucleic acid (or a set of nucleic acids) encoding the antibodies described herein may further comprise a nucleic acid sequence encoding a peptide tag (such as protein purification tag, e.g., His-tag, HA tag).

Also contemplated here are isolated host cells comprising an anti-TNFR2 antibody, anti-PD-L1 or anti-PD-1 antibody, an isolated nucleic acid encoding the polypeptide components of the anti-TNFR2 antibody, anti-PD-L1 or anti-PD-1 antibody, or a vector comprising a nucleic acid encoding the polypeptide components of the anti-TNFR2 antibody, anti-PD-L1 or anti-PD-1 antibody described herein.

The present application also includes variants of these nucleic acid sequences. For example, the variants include nucleotide sequences that hybridize to the nucleic acid sequences encoding the antibodies of the present application under at least moderately stringent hybridization conditions.

The present application also provides vectors in which a nucleic acid of the present application is inserted.

In brief summary, the expression of an antibody by a natural or synthetic nucleic acid encoding the antibody can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present application may also be used for nucleic acid immunization and gene therapy, using standard gene delivery protocols. Methods for gene delivery are known in the art. See, e.g., U.S. Pat. Nos. 5,399,346; 5,580,859; 5,589,466, incorporated by reference herein in their entireties. In some embodiments, the application provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the application should not be limited to the use of constitutive promoters. Inducible promoters are also contemplated as part of the application. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the antibody is inducible. In some embodiments, a nucleic acid sequence encoding the antibody is operably linked to an inducible promoter, including any inducible promoter described herein.

### Inducible Promoters

The use of an inducible promoter provides a molecular switch capable of turning on expression of the polynucleotide sequence which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262: 1019-1024) and ionizing radiation-regulated elements (e.g., see Manome, Y. et al. (1993) Biochemistry 32: 10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci 21:377-405. In some embodiments, the inducible promoter system for use to express the antibody is the Tet system. In some embodiments, the inducible promoter system for use to express the antibody is the lac repressor system from E. coli.

An exemplary inducible promoter system for use in the present application is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli (See Brown et al., Cell 49:603-612 (1987)). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, (3-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding an antibody according to any of the antibodies described herein. In some embodiments, the nucleic acid comprises one or more nucleic acid sequences encoding the heavy and light chains of the antibody. In some embodiments, each of the one or more nucleic acid sequences is contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present application, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the application.

### Preparation of Antibodies

In some embodiments, the antibody (e.g. an antibody that specifically recognizes TNFR2, PD-L1 or PD-1) is a monoclonal antibody. In some embodiments, the antibody is derived from a monoclonal antibody. In some embodiments, the antibody comprises V_{H} and V_{L} domains, or variants thereof, from the monoclonal antibody. In some embodiments, the antibody further comprises C_{H}1 and C_{L} domains, or variants thereof, from the monoclonal antibody. Monoclonal antibodies can be prepared, e.g., using known methods in the art, including hybridoma methods, yeast display, phage display methods, or using recombinant DNA methods. Additionally, exemplary yeast display and phage display methods are described herein and in the Examples below.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the antibodies described herein, the antibody comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phages typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The antibodies can be prepared using phage display to screen libraries for antigen-binding moieties specific to the target antigen (such as TNFR2, PD-L1 or PD-1). The library can be a human scFv phage display library having a diversity of at least 1×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target antigen (such as TNFR2, PD-L1 or PD-1) with high affinity can be selected by iterative binding of phage to the target antigen, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XL1-Blue, for expression and purification. The panning can be performed for multiple (such as about any of 2, 3, 4, 5, 6 or more) rounds with solution panning, cell panning, or a combination of both, to enrich for phage clones binding specifically to the target antigen. Enriched phage clones can be tested for specific binding to the target antigen by any methods known in the art, including for example ELISA and FACS.

Monoclonal antibodies and bispecific molecules can also be made by recombinant DNA methods, such as those described in U.S. Pat. No. 4,816,567. DNA encoding the monoclonal antibodies of the application can be readily isolated and sequenced using conventional procedures (e.g., by using oligonucleotide probes that are capable of binding specifically to genes encoding the heavy and light chains of murine antibodies). Hybridoma cells as described above or antigen-specific phage clones of the application can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the application, or can be substituted for the variable domains of one antigen-combining site of an antibody of the application to create a chimeric bivalent antibody. In some embodiments, additional variable domains targeting a different epitope or antigen can be included to generate a chimeric bispecific antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism. In some embodiments, antibody variable domains targeting different epitopes or different antigens can be fused to immunoglobulin constant-domain sequences to generate a chimeric bispecific molecule.

### Human and Humanized Antibodies

The antibodies can comprise humanized antibody moieties or human antibody moieties. Humanized forms of non-human (e.g., murine) antibody moieties are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody nor in the imported CDR or framework sequences. In general, the humanized antibody can comprise substantially at least one, and typically two, variable domains, in which all or substantially all of the CDR regions correspond to those of a non-human immunoglobulin, and all or substantially all of the FR regions are those of a human immunoglobulin consensus sequence.

Generally, a humanized antibody or humanized antibody moiety has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies or human antibody moieties may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Anti-TNFR2, Anti-PD-L1 or Anti-PD-1 Antibody Variants

In some embodiments, amino acid sequences of the antibodies (e.g., antibody specifically recognizing TNFR2, PD-L1 or PD-1) variants provided herein are contemplated. For example, it may be desirable to improve the binding affinity and/or other biological properties of the antibody. Amino acid sequences of antibody variants may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, variants of the antibodies having one or more amino acid substitutions are provided. Sites of interest for substitutional mutagenesis include the HVRs and FRs. Amino acid substitutions may be introduced into an antibody of interest and the products screened for a desired activity, e.g., improved bioactivity, retained/improved antigen binding, decreased immunogenicity. In some embodiments, the amino acid substitutions described herein are limited to “exemplary substitutions” shown in Table A of this application. In some embodiments, the amino acid substitutions are limited to “preferred substitutions” shown in Table A of this application.

Conservative substitutions are shown in Table A below

TABLE A
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

An exemplary substitutional variant is an affinity matured antibody, which may be conveniently generated, e.g., using phage display-based affinity maturation techniques. Briefly, one or more CDR residues are mutated and the variant antibody moieties displayed on phage and screened for a particular biological activity (e.g., bioactivity based on RBC lysis inhibition assay or binding affinity). Alterations (e.g., substitutions) may be made in HVRs, e.g., to improve bioactivity based on RBC lysis inhibition assay or antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} and V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).)

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., Ala or Glu) to determine whether the interaction of the antibody with antigen is affected. Further substitutions may be introduced at the amino acid locations to demonstrate functional sensitivity to the initial substitutions. Alternatively, or additionally, a crystal structure of an antigen-antibody complex can be determined to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antigen-binding moiety with an N-terminal methionyl residue. Other insertional variants of the antigen-binding moiety include the fusion to the N- or C-terminus of the antigen-binding moiety to an enzyme (e.g. for ADEPT) or a polypeptide which increases the serum half-life of the antigen-binding moiety.

### Fc Region Variants

In some embodiments, one or more amino acid modifications may be introduced into the Fc region of an antibody (e.g., an anti-TNFR2, anti-PD-L1 or anti-PD-1 antibody) provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al. J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., an infected cell), whose membrane-surface antigens have been bound by specific antibodies (e.g., an anti-TNFR2, anti-PD-L1 or anti-PD-1 antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis.

In some embodiments, the application contemplates an antibody (e.g., an anti-TNFR2, anti-PD-L1 or anti-PD-1 antibody) comprising an Fc region that possesses some but not all effector functions, which makes it a desirable candidate for applications in which the half-life of the antibodies in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and 30 FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, alterations are made in the Fc region that result in altered (i.e., either improved or diminished) opsonization, e.g. such as described in Moore et al., MAbs. 2(2): 181-189 (2010).

In some embodiments, there is provided an anti-TNFR2 antibody, anti-PD-L1 or anti-PD-1 antibody comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fe region residues: 238, 256, 265, 272, 286, 303, 305, 307, 311, 312, 317, 340, 356, 360, 362, 376, 378, 380, 382, 413, 424 or 434, e.g., substitution of Fe region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-TNFR2, anti-PD-L1 or anti-PD-1 antibodies (such as full-length anti-TNFR2, anti-PD-L1 or anti-PD-1 antibodies) comprising any of the Fc variants described herein, or combinations thereof, are contemplated.

### Glycosylation Variants

In some embodiments, an anti-TNFR2, anti-PD-L1 or anti-PD-1 antibody provided herein is altered to increase or decrease the extent to which the anti-TNFR2, anti-PD-L1 or anti-PD-1 antibody is glycosylated. Addition or deletion of glycosylation sites to an antibody may be conveniently accomplished by altering the amino acid sequence of the antibody or polypeptide portion thereof such that one or more glycosylation sites is created or removed.

Where the anti-TNFR2, anti-PD-L1 or anti-PD-1 antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the application may be made in order to create an antibody variant with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al., J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-TNFR2, anti-PD-L1 or anti-PD-1 antibody variants are provided comprising an Fe region wherein a carbohydrate structure attached to the Fc region has reduced fucose or lacks fucose, which may improve ADCC function. Specifically, antibodies are contemplated herein that have reduced fucose relative to the amount of fucose on the same antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-TNFR2, anti-PD-L1 or anti-PD-1 antibody is one wherein less than about 50%, 40%, 30%, 20%, 10%, or 5% of the N-linked glycans thereon comprise fucose. For example, the amount of fucose in such an anti-TNFR2, anti-PD-L1 or anti-PD-1 antibody may be from 1% to 80%, from 1% to 65%, from 5% to 65% or from 20% to 40%. In some embodiments, the anti-TNFR2, anti-PD-L1 or anti-PD-1 antibody is one wherein none of the N-linked glycans thereon comprise fucose, i.e., wherein the anti-TNFR2, anti-PD-L1 or anti-PD-1 antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such asα-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-TNFR2, anti-PD-L1 or anti-PD-1 antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-TNFR2, anti-PD-L1 or anti-PD-1 antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-TNFR2, anti-PD-L1 or anti-PD-1 antibody variants comprising an Fc region are capable of binding to an FcγRIII. In some embodiments, the anti-TNFR2, anti-PD-L1 or anti-PD-1 antibody variants comprising an Fc region have ADCC activity in the presence of human effector cells (e.g., T cell) or have increased ADCC activity in the presence of human effector cells compared to the otherwise same antibody comprising a human wild-type Fc region.

### Cysteine Engineered Variants

In some embodiments, it may be desirable to create cysteine engineered anti-TNFR2, anti-PD-L1 or anti-PD-1 antibodies in which one or more amino acid residues are substituted with cysteine residues. In some embodiments, the substituted residues occur at accessible sites of these antibodies. By substituting those residues with cysteine, reactive thiol groups are thereby positioned at accessible sites of the anti-TNFR2 antibody, anti-PD-L1 or anti-PD-1 antibody and may be used to conjugate these antibodies to other moieties, such as drug moieties or linker-drug moieties, to create an anti-TNFR2, anti-PD-L1 or anti-PD-1 antibody immunoconjugate, as described further herein. Cysteine engineered antibodies may be generated as described, e.g., in U.S. Pat. No. 7,521,541.

### Derivatives

In some embodiments, an anti-TNFR2, anti-PD-L1 or anti-PD-1 antibody provided herein may be further modified to contain additional non-proteinaceous moieties that are known in the art and readily available. The moieties suitable for derivatization of the antibodies include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibodies may vary, and if more than one polymer are attached, they can be the same or different molecules. In general, the number and/or type of polymers used for derivatization can be determined based on considerations including, but not limited to, the particular properties or functions of the anti-TNFR2, anti-PD-L1 or anti-PD-1 antibody to be improved, whether the antibody derivative will be used in a therapy under defined conditions, etc.

### Articles of Manufacture and Kits

In some embodiments of the application, there is provided an article of manufacture containing materials useful for the treatment of cancer or infectious diseases. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-TNFR2 antibody, anti-PD-L1, or anti-PD-1 antibody of the application. The label or package insert indicates that the composition is used for treating the particular condition. The label or package insert will further comprise instructions for administering the anti-TNFR2 and anti-PD-L1 or anti-PD-1 antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating cancer or infectious diseases. In some embodiments, the package insert indicates that the composition is used for treating disease or condition selected from the group consisting of non-small cell lung cancer, adrenal gland cancer, bladder cancer, brain cancer, pancreatic adenocarcinoma, breast cancer, colorectal cancer, melanoma, gastroesophageal junction adenocarcinoma, esophageal cancer, esophageal adenocarcinoma, gall bladder cancer, gastric cancer, cervical cancer, gastric adenocarcinoma, head and neck cancer, heart cancer, hepatocellular carcinoma, kidney cancer, liver cancer, mesothelioma, ovarian cancer, pancreatic cancer, prostate cancer, prostate adenocarcinoma, spleen cancer, small cell lung cancer, testicular cancer, thyroid cancer, uterine cancer, and infectious diseases, including, but not limited to Human Papilloma Virus (HPV), Human Immunodeficiency Virus (HIV), Herpes Simplex Virus (HSV), Varicella Zoster Virus (VSV), Cytomegalovirus (CMV), Epstein Barr Virus (EBV), E. coli, Salmonella, Shigella, Staphylococcus aureus, Coliform Bacteria, Chlamydia, Mycobacterium Tuberculosis, Streptococcus, Pneumococcus, Pseudomonas, Campylobacter, Salmonella, Aspergillus Fumigatus, Aspergillus flavus, Cryptococcus Neoformans, and Histoplasma Capsulatum.

Additionally, the article of manufacture may further comprise a second container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

Kits are also provided that are useful for various purposes, e.g., for treatment of disease or condition associated with cancer or infectious diseases, optionally in combination with the articles of manufacture. Kits of the application include one or more containers comprising anti-TNFR2 antibody and anti-PD-L1 or anti-PD-1 antibody composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the application are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-TNFR2 antibody and anti-PD-L1 or anti-PD-1 antibody. In some embodiments, the kit comprises a) any one of the compositions described herein, and b) an effective amount of at least one other agent, wherein the other agent enhances the effects (e.g., treatment effect, detecting effect) of the composition. In some embodiments, the kit comprises a) any one of the compositions described herein, and b) instructions for administering the composition to an individual for treatment of cancer or infectious diseases. In some embodiments, the kit comprises a) any one of the compositions described herein, b) an effective amount of at least one other agent, wherein the other agent enhances the effect (e.g., treatment effect, detecting effect) of the composition, and c) instructions for administering the composition and the other agent(s) to an individual for treatment of cancer or infectious diseases. The anti-TNFR2 antibody and anti-PD-L1 or anti-PD1 antibody composition and the other agent(s) can be present in separate containers or in a single container. For example, the kit may comprise one distinct composition or two or more compositions wherein one composition comprises anti-TNFR2 antibody and anti-PD-L1 or anti-PD-1 antibody and another composition comprises another agent.

In some embodiments, the kit comprises a nucleic acid (or a set of nucleic acids) encoding an anti-TNFR2 antibody. In some embodiments, the kit comprises a) a nucleic acid (or a set of nucleic acids) encoding an anti-TNFR2 antibody, and b) a host cell for expressing the nucleic acid (or a set of nucleic acids). In some embodiments, the kit comprises a) a nucleic acid (or a set of nucleic acids) encoding an anti-TNFR2 antibody, and b) instructions for i) expressing the anti-TNFR2 antibody in a host cell, ii) preparing a composition comprising the anti-TNFR2 antibody, and iii) administering the composition comprising the anti-TNFR2 antibody to an individual for the treatment of cancer or infectious diseases. In some embodiments, the kit comprises a) a nucleic acid (or a set of nucleic acids) encoding an anti-TNFR2 antibody, b) a host cell for expressing the nucleic acid (or a set of nucleic acids), and c) instructions for i) expressing the anti-TNFR2 antibody in the host cell, ii) preparing a composition comprising the anti-TNFR2 antibody, and iii) administering the composition comprising the anti-TNFR2 antibody to an individual for the treatment cancer or infectious diseases.

In some embodiments, the kit comprises a nucleic acid (or a set of nucleic acids) encoding an anti-PD-L1 or PD-1 antibody. In some embodiments, the kit comprises a) a nucleic acid (or a set of nucleic acids) encoding an anti-PD-L1 or PD-1 antibody, and b) a host cell for expressing the nucleic acid (or a set of nucleic acids). In some embodiments, the kit comprises a) a nucleic acid (or a set of nucleic acids) encoding an anti-PD-L1 or PD-1 antibody, and b) instructions for i) expressing the anti-PD-L1 or PD-1 antibody in a host cell, ii) preparing a composition comprising the anti-PD-L1 or PD-1 antibody, and iii) administering the 10 composition comprising the anti-PD-L1 or PD-1 antibody to an individual for the treatment of cancer or infectious diseases. In some embodiments, the kit comprises a) a nucleic acid (or a set of nucleic acids) encoding an anti-PD-L1 or PD-1 antibody, b) a host cell for expressing the nucleic acid (or a set of nucleic acids), and c) instructions for i) expressing the anti-PD-L1 or PD-1 antibody in the host cell, ii) preparing a composition comprising the anti-PD-L1 or PD-1 antibody, and iii) administering the composition comprising the anti-PD-L1 or PD-1 antibody to an individual for the treatment cancer or infectious diseases.

The kits of the application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions comprising anti-TNFR2 antibody and anti-PD-L1 or anti-PD-1 antibody generally include information as to dosage, dosing schedule, and route of administration for the intended treatment. The containers may be unit doses, bulk packages (e.g., multi-dose packages) or sub-unit doses. For example, kits may be provided that contain sufficient dosages of the composition as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this application. The application will now be described in greater detail by reference to the following non-limiting examples. The following examples further illustrate the application but, of course, should not be construed as in any way limiting its scope.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of Anti-TNFR2 Antibodies

This example illustrates the methods of generating anti-TNFR2 antibodies, and methods to screen and select antibodies for further characterization. The content of patent with international application No. PCT/US2022/073523 was incorporated here in this invention.

Immunizations and fusions: Balb/c and NZB mice were immunized with recombinant ECD of human TNFR2 (SEQ ID NO: 25) fused with His- or mouse IgG2a Fc produced in Expi293 or CHO cells adjuvanted with RIBI (Sigma Aldrich, cat #56322-1VL), Titermax (Sigma Aldrich, cat #T2684-1 ML), or/and Freund's (Freund's adjuvant, incomplete) (Sigma Aldrich, cat #F5506-10x-10 mL). Three days after the last immunization, spleens and lymph nodes were harvested and processed according to standard protocols. Mouse B cells were isolated by EasySep Mouse B cell isolation Kit (StemCell, cat #19854A) and fused with myeloma cells SP2/0-Ag14 cells (ATCC, CRL 1581) using PEG. Following standard protocols, the fused cells were plated into six-well plates in semi-solid ClonalCell-HY Cloning-Medium D (StemCell, cat #03804). Monoclonal hybridoma clones were picked into 96 well/plate using Clone Pix 2 Machine (Molecular Devices) and cultured in HT medium.

Hybridoma Screening: After 10-14 days of culture, supernatants were collected and subjected to primary screening by ELISA with 96 well ELISA plates coated with human or cynomolgus monkey TNFR2 extracellular domain (SEQ ID NO: 27) proteins with His or human Fc tag. The parental hybridoma hits identified from the primary screen were expanded. The supernatants of the hybridoma hits identified in the primary screen were further tested for their ability to block the biochemical binding between human TNFR2 and human TNFα. The hybridomas screened were prioritized for subcloning and further characterization.

Purification of hybridoma antibodies: Positive clone 51B5 was scaled up and the antibody was purified using protein A resin. Then the positive clone 51B5 was sequenced and amplified, the sequence of which was shown in Tables 2-3.

Generation of recombinant IgG1 version of 51B5 chimeric antibody: the recombinant 51B5 chimeric antibody construct with heavy and light chain variable domain of mouse antibody and human constant regions were made by using methods well known in the field. The exemplary human heavy chain constant region and light chain constant region were shown in Table 4. The binding affinity and the activity of blocking TNFα binding to TNFR2 of 51B5 chimeric antibody were determined by the experiments described below.

Preparation of humanized versions of the hybridoma clone 51B5: The light chain variable region (VL) and heavy chain variable region (VH) sequences of murine antibody from hybridoma 51B5 were aligned to human germline antibody sequences respectively. The human germline kappa light chain and heavy chain were used as the human frameworks.

The complementarity-determining regions (CDRs) of murine TNFR2 antibody 51B5 light chain and heavy chain were grafted into the identified closest human frameworks respectively to generate humanized antibody clone. In this process, antibody 51B5 was humanized by grafting the CDRs from the murine antibody V-regions onto human germline antibody V-region frameworks, the CDRs grafted from the donor to the acceptor sequence are as defined by Kabat (Kabat et al., 1987). In order to recover the activity of the antibody, a number of framework residues from the murine V-regions that were found to be parts of VH-VL interacting interface or the framework residues acting as “Vernier” zone, which may adjust CDR structure and fine-tune to fit to antigen (Foote et al., 1992) were also retained in the humanized sequence.

The sequences of the humanized antibodies were summarized in Tables 2-3.

### Example 2: Characterization and Activity Evaluation of Anti-TNFR2 Antibodies Epitope Mapping by Alanine Scanning Assay

It had been identified the TNFR2 antibody 51B5 was bound to CRD3 of human TNFR2 receptor. In this assay, the epitope on human TNFR2 for humanized anti-TNFR2 antibody SB1901-19, SB1901-72, or SB1901-76 was mapped by alanine scanning. The alanine scanning mutations within huTNFR2 CRD3 region of the chimeric protein musTNFR2-huCRD3(ECD) (SEQ ID NO: 26) were generated. The His-tag was added to the C terminus of the chimeric protein mutations for purification and detection. A plate was coated with the humanized TNFR2 antibody SB1901-19, SB1901-72, or SB1901-76 overnight at 4° C. After washing, the alanine mutations of the chimeric proteins were added and incubated with shaking for 2 hours at room temperature. The plate was washed again. Then, anti-His antibody conjugated with AP was added to the plate and incubated for 1 hour at room temperature. The plate was washed and developed with pNPP substrate for 30 min. Plates were read at 450 nm wavelength.

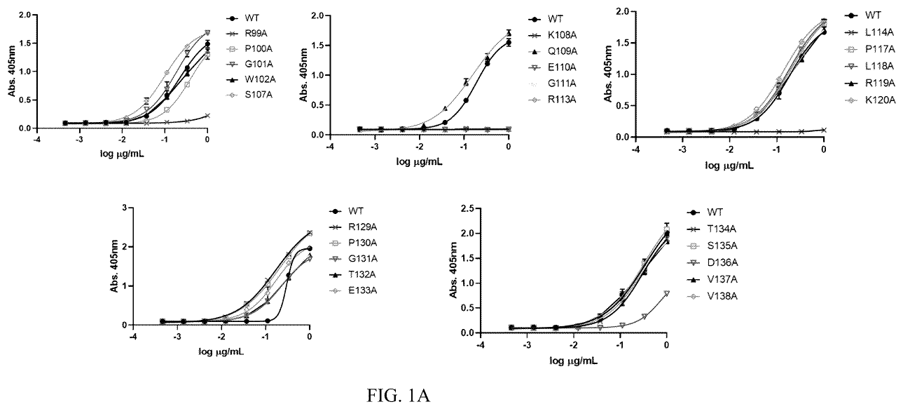

The alanine scanning results showed that the epitope of the humanized anti-TNFR2 antibody SB1901-19, SB1901-72, or SB1901-76 was the same (data not shown). FIG. 1A-1B showed the alanine scanning results of exemplary antibody SB1901-76, and the amino acids R99, K108, E110, G111, R113, L114, and D136 in the CRD3 region of human TNFR2 (SEQ ID NO 25) are required for the antibody binding to human TNFR2. The results indicated that the conformational epitope of SB1901-19, SB1901-72, or SB1901-76 antibody comprises or consists of the amino acid residues R99, K108, E110, G111, R113, L114, and D136 according to SEQ ID NO 25.

### Binding Affinities

Binding affinities (monovalent Kd) of anti-TNFR2 antibodies were determined using Biolayer interferometry on the Octet RED96 instrument (ForteBio) at 30° C. and with 1200 rpm agitation. The following kinetic assay was performed using anti-human IgG Fc capture (AHC) biosensors (ForteBio) in kinetics buffer (PBS, 0.1% Tween-20 and 1% bovine serum albumin): (a) antibody (2 μg/mL) for 300 sec, (b) baseline for 120 sec, (c) association with His-tag-huTNFR2 (2.5, 0.5 and 0 μg/mL) for 420 sec and (d) dissociation for 1200 sec. Data fitting and analysis was performed with Octet data analysis software 8.0 (ForteBio) using a 1:1 binding model after Savitzky-Golay filtering. The dissociation constant (Kd) was calculated as the ratio of Koff/Kon. Examples of binding affinity of humanized antibodies were shown in Table 5.

TABLE 5
Binding affinity of humanized antibodies to TNFR2 antigens

| Antibody | Kd (nM) | Kon (1/Ms) | Koff (1/s) |
|---|---|---|---|
| SB1901-72 | 1.11 | 4.12E+05 | 4.56E−04 |
| SB1901-80 | 2.52 | 3.73E+05 | 9.37E−04 |

Binding to huTNFR2 Expressed Cells

To examine the binding of anti-TNFR2 antibodies to huTNFR2 expressed cells, Expi293 cells stably expressing huTNFR2 were used to perform FACS analysis.

The coding sequence of huTNFR2 (Uniprot, P20333) was cloned into a lentiviral vector and the virus was packaged according to the instruction of the virus packaging kit (Lenti-X™ Packaging Single Shots, Cat #631275, Takada). The Expi293 cells were transduced with the recombinant virus and selected by puromycin. The cell line stably expressing huTNFR2 was incubated with anti-TNFR2 antibodies in PBS with 0.5% BSA, 1 mM EDTA, and 0.1% sodium azide (FACS buffer) for 30 minutes at 4° C. The cells were washed, and then incubated with 10 nM phycoerythrin (PE) conjugated anti-Human Fc Ab (Biolegend, cat #409304) for 20 minutes at 4° C. Cells were washed and then isolated by flow cytometry with Attune (ThermoFisher Scientific). Data were analyzed with FlowJo software. Antibody binding is represented as median fluorescence intensity (MFI).

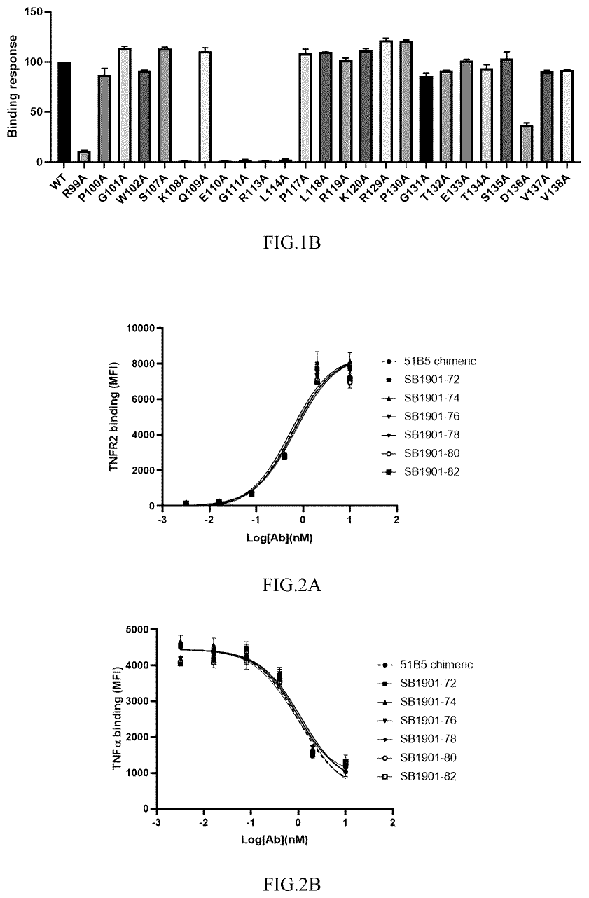

As shown in FIG. 2A, the chimeric anti-TNFR2 antibody 51B5 and humanized antibodies SB1901-72, SB1901-74, SB1901-76, SB1901-78, SB1901-80 and SB1901-82 could bind to Expi293-TNFR2 cells dose-dependently and potently.

Binding to huTNFR2-Expi293 Cells and Blocking of TNFα Binding

Expi293 cells stably expressing huTNFR2 were incubated with anti-TNFR2 antibodies for 30 minutes at 4° C. The cells were washed, and then incubated with 10 nM Alexa Fluor 647 conjugated (ThermoFisher Scientific, cat #A20186) human TNFα (SinoBiological, cat #10602-HNAE) for 20 minutes at 4° C. Cells were washed and acquired by flow cytometry with Attune. Data were analyzed with FlowJo software. TNFα binding is represented as MFI.

As shown in FIG. 2B, the chimeric anti-TNFR2 antibody 51B5 and humanized antibodies SB1901-72, SB1901-74, SB1901-76, SB1901-78, SB1901-80 and SB1901-82 inhibited soluble TNFα binding to TNFR2-expressing Expi293 cells dose-dependently and potently.

### In Vitro Human Primary Treg Cell Proliferation Assay

Functional activity of the anti-TNFR2 antibodies on human primary Treg cells was tested. PBMCs were incubated with 200 U/ml IL-2 and 20 ng/ml TNFα in the presence or absence of anti-TNFR2 antibodies in complete media in round bottom plates at 37° C. for 72 hours. Cells were stained with anti-CD3 antibody and anti-CD4 antibody in FACS buffer for 30 minutes at 4° C. Cells were washed and fixed/permeabilized with fix/permeabilize buffer for 30 minutes at 4° C. They were then washed with 1× permeabilization buffer and stained with anti-human Foxp3 antibody in 1× permeabilization buffer for 30 minutes at 4° C. Cells were washed, fixed with 2% PFA, isolated and analyzed by flow cytometry with Attune. The percentage of Foxp3+ cells in CD4+ cells was analyzed with FlowJo software.

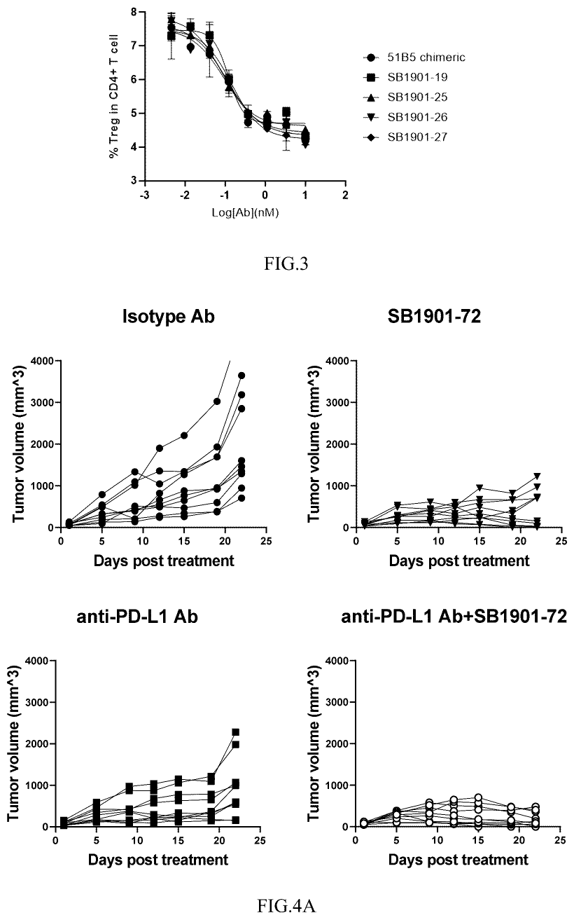

As shown in FIG. 3, the chimeric anti-TNFR2 antibody 51B5 and the exemplary humanized TNFR2 antibodies SB1901-19, SB1901-25, SB1901-26 and SB1901-27 could inhibit the Treg cell proliferation in vitro.

### Example 3: In Vivo Anti-Tumor Efficacy Study to Evaluate Activity of the Combination of Anti-TNFR2 Antibody and Anti-PDL1 Antibody

This example illustrated in vivo tumor model study of the functional activity of the humanized anti-TNFR2 antibody as a single agent or in combination with anti-PD-L1 or anti-PD-1 antibody.

Subcutaneous Tumor Model: MC38 was used herein.

Animals and husbandry: Forty female C57BL/6-Tnfrsf1btm1 (TNF-RSF1B)/Bcgen mice (6-9 weeks of age) were used in the studies. The animals were fed breeding diet for “SPF rat and mouse growth” and water ad libitum. Animals were ear tagged for identification purposes and shaved on the left dorsal flank area in preparation of cell implantation. Animals were housed in polycarbonate cages (cage size of 320×200×135 mm). The environment was controlled to a temperature range of 20° C.-26° C. and a humidity range of 40-70%. Animal care and use were compliant with the SOPs of JOINN LABORATORIES (Suzhou) Inc., the Guide for the Care and Use of Laboratory Animals (8th Edition, Institute of Laboratory Animal Resources, Commission on Life Sciences, National Research Council; National Academy Press; Washington, D.C., 2010), and the U.S. Department of Agriculture through the Animal Welfare Act (Public Law 99-198).

Cell preparation and implantation: Mouse colon cancer cell line MC38, purchased from Institute of Basic Medical Sciences were cultured and expanded in RPMI medium with 2 mM L-glutamine, 10% fetal bovine serum (FBS), and 1% 100× Penicillin/Streptomycin (PS). The growth environment was maintained in an incubator with a 5% CO2 atmosphere at 37° C. When expansion was complete, the cells (passage 3) were trypsinized using a 0.25% trypsin-EDTA solution. The cells were then washed and counted. Pre-implantation cell viability was 92%-94%. The cells were suspended in Dulbecco's Phosphate Buffered Saline (DPBS) at a concentration of 1×10⁷/ml. Test animals were sterilized at the implantation site with an alcohol prep pad and were implanted subcutaneously in 0.2 mL using a 25-gauge needle and 1 mL syringe.

Measurements and antibody treatment: Tumors were allowed to grow and mice were then randomized into study groups. Mice were distributed to ensure that the mean body weights for all groups were within 10% of the overall mean tumor burden for the study population. Human MOPC21 IgG1 isotype antibody (see Hamlyn PH, Gait MJ, Milstein C. (1981) Complete sequence of an immunoglobulin mRNA using specific priming and the dideoxynucleotide method of RNA sequencing. Nucleic Acids Res. 9(18):4485-4494), SB1901-72 and anti-PD-L1 antibody (Atezolizumab, Genentech) were made in-house. Anti-PD-1 antibody (Clone No. RMP1-14) was purchased from BioXcell. Mice received twice weekly i.p. injections of antibody treatment for 3 weeks and tumor volumes were monitored (n=10 mice/group). The major axis and minor axis of tumors were measured with a vernier caliper and recorded to calculate the tumor volume, and the tumor growth curve was drawn according to the tumor volume to compare the differences between the groups. Tumor volume was calculated according to the following formula: V=½× major axis× minor axis².

Assessment of side effects: All animals were observed for clinical signs of distress or toxicity at least once daily. Animals were weighed once per week. Animals were euthanized if body weight loss was in excess of 20% or other clinical signs that warranted euthanasia. Individual animals were euthanized when their tumor volume reached or exceeded 2500 mm³.

Results: The tumor volume changes and the average tumor growth volume in each group were calculated.

Combination Therapy with Anti-TNFR2 Antibody and Anti-PD-L1 Antibody

The study summary of combination therapy with antibody SB1902-72 and an anti-PD-L1 antibody in MC38 tumor model was shown in Table 6. MC38 tumors in human TNFR2 transgenic mice were treated intraperitoneally with SB1901-72 (10 mg/kg), anti-PD-L1 Ab (5 mg/kg), the combination of the former two, or isotype antibody (10 mg/kg) twice a week for 6 injections. Tumor size was monitored twice per week.

TABLE 6
Summary of in vivo subcutaneous tumor study protocol

| | Strain | Tumor Volume | | | |
|---|---|---|---|---|---|
| Model | (Vendor) | at randomization | Treatment Groups | | Dosing |
| MC38 | C57BL/6- | 33-156 mm³ | Control | Human MOPC21 | 10 mg/kg |
| | Tnfrsf1b^{tm1 (TNF-RSF1B)}/ | | group | isotype antibody | |
| | Bcgen (Biocytogen) | | Group 1 | SB1901-72 | 10 mg/kg |
| | | | Group 2 | Anti-PD-L1 antibody | 5 mg/kg |
| | | | Group 3 | Anti-PD-L1 antibody + | 5 mg/kg + |
| | | | | SB1901-72 | 10 mg/kg |

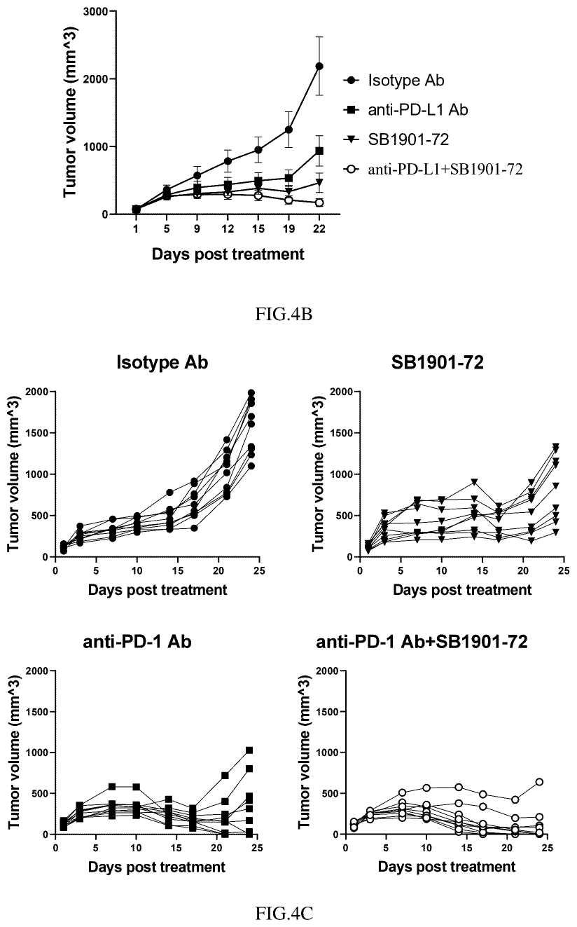

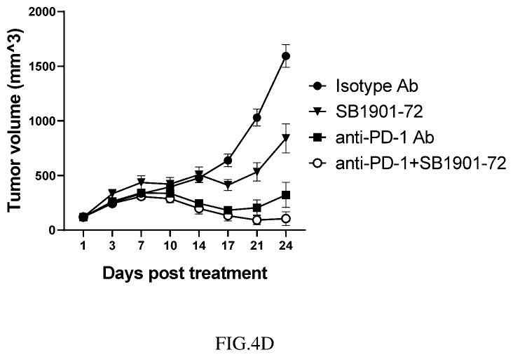

Tumor growth curves of the individual mouse in monotherapies or in combination therapy were shown in FIG. 4A, and the average tumor growth curve of each group was shown in FIG. 4B. The results showed that the tumor volume in all treated groups were significantly lower than that in isotype antibody treated group (P<0.001) on Day 22, indicating that TNFR2 blockade by SB1901-72 efficiently suppressed tumor growth. In addition, the combination of the anti-PD-L1 antibody with SB1901-72 shows the trend of being more effective than the single antibody treatment. There were no significant differences in body weights between the groups at the same time point. The animals did not show abnormality in general clinical observation.

Combination Therapy with Anti-TNFR2 Antibody and Anti-PD-1 Antibody

The study summary of combination therapy with antibody SB1902-72 and anti-PD-1 antibody in MC38 tumor model was shown in Table 7. MC38 tumors in human TNFR2 transgenic mice were treated intraperitoneally with SB1901-72, anti-PD-1 Ab, the combination of the former two, or isotype antibody at 3 mg/kg, twice a week for 6 injections. Tumor size was monitored twice per week.

TABLE 7
Summary of in vivo subcutaneous tumor study protocol

| | Strain | Tumor Volume | | | |
|---|---|---|---|---|---|
| Model | (Vendor) | at randomization | Treatment Groups | | Dosing |
| MC38 | C57BL/6- | 70-150 mm³ | Control | Human MOPC21 | 3 mg/kg |
| | Tnfrsf1b^{tm1 (TNF-RSF1B)}/ | | group | isotype antibody | |
| | Bcgen (Biocytogen) | | Group 1 | SB1901-72 | 3 mg/kg |
| | | | Group 2 | Anti-PD-1 antibody | 3 mg/kg |
| | | | Group 3 | Anti-PD-1 antibody + | 3 mg/kg + |
| | | | | SB1901-72 | 3 mg/kg |

Tumor growth curves of the individual mouse in monotherapies or in combination therapy were shown in FIG. 4C, and the average tumor growth curve of each group was shown in FIG. 4D. The results showed that the tumor growth in all treated groups was significantly suppressed compared to that in isotype antibody treated group (P<0.001), and it can be seen from the tumor growth curve of the individual mouse in each group that tumors disappeared in 4/10 mice in anti-PD-1 Ab group vs. 8/10 mice in the combination therapy group. The combination of the anti-PD-1 antibody with SB1901-72 was more effective than the single antibody therapy.

In conclusion, these results indicated that the combination of the anti-TNFR2 antibody 10 (SB1901-72) with the anti-PD-1 or anti-PD-L1 antibody enhanced antitumor effects compared with the single antibody.

1. A method of treating cancer or infectious disease in an individual in need thereof, comprising administering to the individual an antibody specifically recognizing human TNFR2 and an antibody specifically recognizing human PD-L1 or PD-1, wherein the anti-TNFR2 antibody comprises: a heavy chain variable domain (V_{H}) comprising a heavy chain complementarity determining region (HC-CDR) 1 comprising DDYID (SEQ ID NO: 1), an HC-CDR2 comprising EIYPGSGNTYYNEKFKG (SEQ ID NO: 2), and an HC-CDR3 comprising SQVYGKIAMDH (SEQ ID NO: 3);
and a light chain variable domain (V_{L}) comprising a light chain complementarity determining region (LC-CDR) 1 comprising RASESVDNSGNSFMH (SEQ ID NO: 4), a LC-CDR2 comprising RASNLES (SEQ ID NO: 5), and a LC-CDR3 comprising QQSKEDPYT (SEQ ID NO: 6).

2-3. (canceled)

4. The method of claim 1, wherein the antibody specifically recognizing human TNFR2 and the antibody specifically recognizing human PD-L1 or PD-1 are administered to the individual in need thereof concurrently or sequentially.

5. A pharmaceutical composition comprising an antibody specifically recognizing human TNFR2 and an antibody specifically recognizing human PD-L1 or PD-1, wherein the anti-TNFR2 antibody comprises: a heavy chain variable domain (V_{H}) comprising a heavy chain complementarity determining region (HC-CDR) 1 comprising DDYID (SEQ ID NO: 1), an HC-CDR2 comprising EIYPGSGNTYYNEKFKG (SEQ ID NO: 2), and an HC-CDR3 comprising SQVYGKIAMDH (SEQ ID NO: 3);
and a light chain variable domain (V_{L}) comprising a light chain complementarity determining region (LC-CDR) 1 comprising RASESVDNSGNSFMH (SEQ ID NO: 4), a LC-CDR2 comprising RASNLES (SEQ ID NO: 5), and a LC-CDR3 comprising QQSKEDPYT (SEQ ID NO: 6).

6. A combination of an antibody specifically recognizing human TNFR2 and an antibody specifically recognizing human PD-L1 or PD-1, wherein the anti-TNFR2 antibody comprises: a heavy chain variable domain (V_{H}) comprising a heavy chain complementarity determining region (HC-CDR) 1 comprising DDYID (SEQ ID NO: 1), an HC-CDR2 comprising EIYPGSGNTYYNEKFKG (SEQ ID NO: 2), and an HC-CDR3 comprising SQVYGKIAMDH (SEQ ID NO: 3);
and a light chain variable domain (V_{L}) comprising a light chain complementarity determining region (LC-CDR) 1 comprising RASESVDNSGNSFMH (SEQ ID NO: 4), a LC-CDR2 comprising RASNLES (SEQ ID NO: 5), and a LC-CDR3 comprising QQSKEDPYT (SEQ ID NO: 6).

7-9. (canceled)

10. The method of claim 1 wherein the anti-PD-1 antibody is selected from the group consisting of cemiplimab, nivolumab, pembrolizumab, sintilimab, camrelizumab, toripalimab, tislelizumab, zimberelimab, prolgolimab, dostarlimab, AMG-404, balstilimab, budigalimab, cetrelimab, ezabenlimab, genolimzumab, LZM-009, nofazinlimab, penpulimab, pimivalimab, pucotenlimab, QL-1604, retifanlimab, rulonilimab, sasanlimab, SCT-I10A, serplulimab, SG-001, spartalizumab, SYSA-1802 and TY-101.

11. The method of claim 1 wherein the anti-PD-L1 antibody is selected from the group consisting of avelumab, durvalumab, atezolizumab, KN035, CK-301, ADG-104, BCD-135, garivulimab, CBT-502, HLX-20, IMC-001, tagitanlimab, LAE-005, LP-002, MSB-2311, adebrelimab, sugemalimab and socazolimab.

12-14. (canceled)

15. The method of claim 1 wherein the anti-TNFR2 antibody comprises:
(i) a V_{H} comprising the amino acid sequence of SEQ ID NO: 7, or a variant thereof having at least about 80% sequence identity to SEQ ID NO: 7 and a V_{L} comprising the amino acid sequence of SEQ ID NO: 16, or a variant thereof having at least about 80% sequence identity to SEQ ID NO: 16;
(ii) a V_{H} comprising the amino acid sequence of SEQ ID NO: 8, or a variant thereof having at least about 80% sequence identity to SEQ ID NO: 8 and a V_{L} comprising the amino acid sequence of SEQ ID NO: 17, or a variant thereof having at least about 80% sequence identity to SEQ ID NO: 17;
(iii) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 18, or a variant thereof having at least about 80% sequence identity to SEQ ID NO: 18;
(iv) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 19, or a variant thereof having at least about 80% sequence identity to SEQ ID NO: 19;
(v) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 17, or a variant thereof having at least about 80% sequence identity to SEQ ID NO: 17;
(vi) a V_{H} comprising the amino acid sequence of SEQ ID NO: 10, or a variant thereof having at least about 80% sequence identity to SEQ ID NO: 10 and a V_{L} Comprising the amino acid sequence of SEQ ID NO: 20, or a variant thereof having at least about 80% sequence identity to SEQ ID NO: 20;
(vii) a V_{H} comprising the amino acid sequence of SEQ ID NO: 11, or a variant thereof having at least about 80% sequence identity to SEQ ID NO: 11 and a V_{L} comprising the amino acid sequence of SEQ ID NO: 20, or a variant thereof having at least about 80% sequence identity to SEQ ID NO: 20;
(viii) a V_{H} comprising the amino acid sequence of SEQ ID NO: 12, or a variant thereof having at least about 80% sequence identity to SEQ ID NO: 12 and a V_{L} comprising the amino acid sequence of SEQ ID NO: 20, or a variant thereof having at least about 80% sequence identity to SEQ ID NO: 20;
(ix) a V_{H} comprising the amino acid sequence of SEQ ID NO: 13, or a variant thereof having at least about 80% sequence identity to SEQ ID NO: 13 and a V_{L} comprising the amino acid sequence of SEQ ID NO: 20, or a variant thereof having at least about 80% sequence identity to SEQ ID NO: 20;
(x) a V_{H} comprising the amino acid sequence of SEQ ID NO: 14, or a variant thereof having at least about 80% sequence identity to SEQ ID NO: 14 and a V_{L} comprising the amino acid sequence of SEQ ID NO: 20, or a variant thereof having at least about 80% sequence identity to SEQ ID NO: 20; or
(xi) a V_{H} comprising the amino acid sequence of SEQ ID NO: 15, or a variant thereof having at least about 80% sequence identity to SEQ ID NO: 15 and a V_{L} comprising the amino acid sequence of SEQ ID NO: 20, or a variant thereof having at least about 80% sequence identity to SEQ ID NO: 20.

16. The method of claim 1, wherein:
(i) the anti-TNFR2 antibody further comprises an Fc fragment, or
(ii) the anti-TNFR2 antibody is a full-length IgG antibody, optionally is a full-length IgG1, IgG2, IgG3 or IgG4 antibody; or
(iii) the anti-TNFR2 antibody is chimeric, human, or humanized; or
(iv) the anti-TNFR2 antibody is an antigen binding fragment selected from the group consisting of a Fab, a Fab′, a F(ab)′2, a Fab′-SH, a single-chain Fv (scFv), an Fv fragment, a dAb, a Fd, or a diabody.

17. The method of claim 1, wherein, the anti-TNFR2 antibody comprises:
(i) a heavy chain comprising the amino acid sequence of SEQ ID NO: 28 or a variant thereof having at least about 80% sequence identity to SEQ ID NO: 28; and a light chain comprising the amino acid sequence of SEQ ID NO: 37 or a variant thereof having at least about 80% sequence identity to SEQ ID NO: 37;
(ii) a heavy chain comprising the amino acid sequence of SEQ ID NO: 29 or a variant thereof having at least about 80% sequence identity to SEQ ID NO: 29; and a light chain comprising the amino acid sequence of SEQ ID NO: 38 or a variant thereof having at least about 80% sequence identity to SEQ ID NO: 38;
(iii) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 39 or a variant thereof having at least about 80% sequence identity to SEQ ID NO: 39;
(iv) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 40 or a variant thereof having at least about 80% sequence identity to SEQ ID NO: 40;
(v) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 38 or a variant thereof having at least about 80% sequence identity to SEQ ID NO: 38;
(vi) a heavy chain comprising the amino acid sequence of SEQ ID NO: 31 or a variant thereof having at least about 80% sequence identity to SEQ ID NO: 31; and a light chain comprising the amino acid sequence of SEQ ID NO: 41 or a variant thereof having at least about 80% sequence identity to SEQ ID NO: 41;
(vii) a heavy chain comprising the amino acid sequence of SEQ ID NO: 32 or a variant thereof having at least about 80% sequence identity to SEQ ID NO: 32; and a light chain comprising the amino acid sequence of SEQ ID NO: 41 or a variant thereof having at least about 80% sequence identity to SEQ ID NO: 41;
(viii) a heavy chain comprising the amino acid sequence of SEQ ID NO: 33 or a variant thereof having at least about 80% sequence identity to SEQ ID NO: 33; and a light chain comprising the amino acid sequence of SEQ ID NO: 41 or a variant thereof having at least about 80% sequence identity to SEQ ID NO: 41;
(ix) a heavy chain comprising the amino acid sequence of SEQ ID NO: 34 or a variant thereof having at least about 80% sequence identity to SEQ ID NO: 34; and a light chain comprising the amino acid sequence of SEQ ID NO: 41 or a variant thereof having at least about 80% sequence identity to SEQ ID NO: 41;
(x) a heavy chain comprising the amino acid sequence of SEQ ID NO: 35 or a variant thereof having at least about 80% sequence identity to SEQ ID NO: 35; and a light chain comprising the amino acid sequence of SEQ ID NO: 41 or a variant thereof having at least about 80% sequence identity to SEQ ID NO: 41; or
(xi) a heavy chain comprising the amino acid sequence of SEQ ID NO: 36 or a variant thereof having at least about 80% sequence identity to SEQ ID NO: 36; and a light chain comprising the amino acid sequence of SEQ ID NO: 41 or a variant thereof having at least about 80% sequence identity to SEQ ID NO: 41.

18. The method of claim 1, wherein the cancer or infectious disease is selected from the group consisting of lung cancer, skin cancer, lymphoma, leukemia, adrenal gland cancer, bladder cancer, brain cancer, pancreatic adenocarcinoma, breast cancer, colorectal cancer, melanoma, gastroesophageal junction adenocarcinoma, esophageal cancer, esophageal adenocarcinoma, gall bladder cancer, gastric cancer, cervical cancer, gastric adenocarcinoma, head and neck cancer, heart cancer, hepatocellular carcinoma, kidney cancer, liver cancer, mesothelioma, ovarian cancer, pancreatic cancer, prostate cancer, prostate adenocarcinoma, spleen cancer, small or non-small cell lung cancer, testicular cancer, thyroid cancer, uterine cancer, and infectious diseases, including, but not limited to Human Papilloma Virus (HPV), Human Immunodeficiency Virus (HIV), Herpes Simplex Virus (HSV), Varicella Zoster Virus (VSV), Cytomegalovirus (CMV), Epstein Barr Virus (EBV), E. coli, Salmonella, Shigella, Staphylococcus aureus, Coliform Bacteria, Chlamydia, Mycobacterium Tuberculosis, Streptococcus, Pneumococcus, Pseudomonas, Campylobacter, Salmonella, Aspergillus Fumigatus, Aspergillus flavus, Cryptococcus Neoformans, and Histoplasma Capsulatum.

19. The pharmaceutical composition of claim 5, wherein the anti-TNFR2 antibody comprises:
(i) a V_{H} comprising the amino acid sequence of SEQ ID NO: 7, or a variant thereof having at least about 80% sequence identity to SEQ ID NO: 7 and a V_{L} comprising the amino acid sequence of SEQ ID NO: 16, or a variant thereof having at least about 80% sequence identity to SEQ ID NO: 16;
(ii) a V_{H} comprising the amino acid sequence of SEQ ID NO: 8, or a variant thereof having at least about 80% sequence identity to SEQ ID NO: 8 and a V_{L} comprising the amino acid sequence of SEQ ID NO: 17, or a variant thereof having at least about 80% sequence identity to SEQ ID NO: 17;
(iii) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 18, or a variant thereof having at least about 80% sequence identity to SEQ ID NO: 18;
(iv) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 19, or a variant thereof having at least about 80% sequence identity to SEQ ID NO: 19;
(v) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 17, or a variant thereof having at least about 80% sequence identity to SEQ ID NO: 17;
(vi) a V_{H} comprising the amino acid sequence of SEQ ID NO: 10, or a variant thereof having at least about 80% sequence identity to SEQ ID NO: 10 and a V_{L} Comprising the amino acid sequence of SEQ ID NO: 20, or a variant thereof having at least about 80% sequence identity to SEQ ID NO: 20;
(vii) a V_{H} comprising the amino acid sequence of SEQ ID NO: 11, or a variant thereof having at least about 80% sequence identity to SEQ ID NO: 11 and a V_{L} comprising the amino acid sequence of SEQ ID NO: 20, or a variant thereof having at least about 80% sequence identity to SEQ ID NO: 20;
(viii) a V_{H} comprising the amino acid sequence of SEQ ID NO: 12, or a variant thereof having at least about 80% sequence identity to SEQ ID NO: 12 and a V_{L} comprising the amino acid sequence of SEQ ID NO: 20, or a variant thereof having at least about 80% sequence identity to SEQ ID NO: 20;
(ix) a V_{H} comprising the amino acid sequence of SEQ ID NO: 13, or a variant thereof having at least about 80% sequence identity to SEQ ID NO: 13 and a V_{L} comprising the amino acid sequence of SEQ ID NO: 20, or a variant thereof having at least about 80% sequence identity to SEQ ID NO: 20;
(x) a V_{H} comprising the amino acid sequence of SEQ ID NO: 14, or a variant thereof having at least about 80% sequence identity to SEQ ID NO: 14 and a V_{L} comprising the amino acid sequence of SEQ ID NO: 20, or a variant thereof having at least about 80% sequence identity to SEQ ID NO: 20; or
(xi) a V_{H} comprising the amino acid sequence of SEQ ID NO: 15, or a variant thereof having at least about 80% sequence identity to SEQ ID NO: 15 and a V_{L} comprising the amino acid sequence of SEQ ID NO: 20, or a variant thereof having at least about 80% sequence identity to SEQ ID NO: 20.

20. The pharmaceutical composition of claim 5, wherein the anti-TNFR2 antibody comprises:
(i) a heavy chain comprising the amino acid sequence of SEQ ID NO: 28 or a variant thereof having at least about 80% sequence identity to SEQ ID NO: 28; and a light chain comprising the amino acid sequence of SEQ ID NO: 37 or a variant thereof having at least about 80% sequence identity to SEQ ID NO: 37;
(ii) a heavy chain comprising the amino acid sequence of SEQ ID NO: 29 or a variant thereof having at least about 80% sequence identity to SEQ ID NO: 29; and a light chain comprising the amino acid sequence of SEQ ID NO: 38 or a variant thereof having at least about 80% sequence identity to SEQ ID NO: 38;
(iii) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 39 or a variant thereof having at least about 80% sequence identity to SEQ ID NO: 39;
(iv) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 40 or a variant thereof having at least about 80% sequence identity to SEQ ID NO: 40;
(v) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 38 or a variant thereof having at least about 80% sequence identity to SEQ ID NO: 38;
(vi) a heavy chain comprising the amino acid sequence of SEQ ID NO: 31 or a variant thereof having at least about 80% sequence identity to SEQ ID NO: 31; and a light chain comprising the amino acid sequence of SEQ ID NO: 41 or a variant thereof having at least about 80% sequence identity to SEQ ID NO: 41;
(vii) a heavy chain comprising the amino acid sequence of SEQ ID NO: 32 or a variant thereof having at least about 80% sequence identity to SEQ ID NO: 32; and a light chain comprising the amino acid sequence of SEQ ID NO: 41 or a variant thereof having at least about 80% sequence identity to SEQ ID NO: 41;
(viii) a heavy chain comprising the amino acid sequence of SEQ ID NO: 33 or a variant thereof having at least about 80% sequence identity to SEQ ID NO: 33; and a light chain comprising the amino acid sequence of SEQ ID NO: 41 or a variant thereof having at least about 80% sequence identity to SEQ ID NO: 41;
(ix) a heavy chain comprising the amino acid sequence of SEQ ID NO: 34 or a variant thereof having at least about 80% sequence identity to SEQ ID NO: 34; and a light chain comprising the amino acid sequence of SEQ ID NO: 41 or a variant thereof having at least about 80% sequence identity to SEQ ID NO: 41;
(x) a heavy chain comprising the amino acid sequence of SEQ ID NO: 35 or a variant thereof having at least about 80% sequence identity to SEQ ID NO: 35; and a light chain comprising the amino acid sequence of SEQ ID NO: 41 or a variant thereof having at least about 80% sequence identity to SEQ ID NO: 41; or
(xi) a heavy chain comprising the amino acid sequence of SEQ ID NO: 36 or a variant thereof having at least about 80% sequence identity to SEQ ID NO: 36; and a light chain comprising the amino acid sequence of SEQ ID NO: 41 or a variant thereof having at least about 80% sequence identity to SEQ ID NO: 41.

21. The pharmaceutical composition of claim 5, wherein the anti-PD-1 antibody is selected from the group consisting of cemiplimab, nivolumab, pembrolizumab, sintilimab, camrelizumab, toripalimab, tislelizumab, zimberelimab, prolgolimab, dostarlimab, AMG-404, balstilimab, budigalimab, cetrelimab, ezabenlimab, genolimzumab, LZM-009, nofazinlimab, penpulimab, pimivalimab, pucotenlimab, QL-1604, retifanlimab, rulonilimab, sasanlimab, SCT-I10A, serplulimab, SG-001, spartalizumab, SYSA-1802 and TY-101.

22. The pharmaceutical composition of claim 5, wherein the anti-PD-L1 antibody is selected from the group consisting of avelumab, durvalumab, atezolizumab, KN035, CK-301, ADG-104, BCD-135, garivulimab, CBT-502, HLX-20, IMC-001, tagitanlimab, LAE-005, LP-002, MSB-2311, adebrelimab, sugemalimab and socazolimab.

23. The combination of claim 6, wherein the anti-TNFR2 antibody comprises:
(i) a V_{H} comprising the amino acid sequence of SEQ ID NO: 7, or a variant thereof having at least about 80% sequence identity to SEQ ID NO: 7 and a V_{L} comprising the amino acid sequence of SEQ ID NO: 16, or a variant thereof having at least about 80% sequence identity to SEQ ID NO: 16;
(ii) a V_{H} comprising the amino acid sequence of SEQ ID NO: 8, or a variant thereof having at least about 80% sequence identity to SEQ ID NO: 8 and a V_{L} comprising the amino acid sequence of SEQ ID NO: 17, or a variant thereof having at least about 80% sequence identity to SEQ ID NO: 17;
(iii) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 18, or a variant thereof having at least about 80% sequence identity to SEQ ID NO: 18;
(iv) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 19, or a variant thereof having at least about 80% sequence identity to SEQ ID NO: 19;
(v) a V_{H} comprising the amino acid sequence of SEQ ID NO: 9, or a variant thereof having at least about 80% sequence identity to SEQ ID NO: 9 and a V_{L} comprising the amino acid sequence of SEQ ID NO: 17, or a variant thereof having at least about 80% sequence identity to SEQ ID NO: 17;
(vi) a V_{H} comprising the amino acid sequence of SEQ ID NO: 10, or a variant thereof having at least about 80% sequence identity to SEQ ID NO: 10 and a V_{L} Comprising the amino acid sequence of SEQ ID NO: 20, or a variant thereof having at least about 80% sequence identity to SEQ ID NO: 20;
(vii) a V_{H} comprising the amino acid sequence of SEQ ID NO: 11, or a variant thereof having at least about 80% sequence identity to SEQ ID NO: 11 and a V_{L} comprising the amino acid sequence of SEQ ID NO: 20, or a variant thereof having at least about 80% sequence identity to SEQ ID NO: 20;
(viii) a V_{H} comprising the amino acid sequence of SEQ ID NO: 12, or a variant thereof having at least about 80% sequence identity to SEQ ID NO: 12 and a V_{L} comprising the amino acid sequence of SEQ ID NO: 20, or a variant thereof having at least about 80% sequence identity to SEQ ID NO: 20;
(ix) a V_{H} comprising the amino acid sequence of SEQ ID NO: 13, or a variant thereof having at least about 80% sequence identity to SEQ ID NO: 13 and a V_{L} comprising the amino acid sequence of SEQ ID NO: 20, or a variant thereof having at least about 80% sequence identity to SEQ ID NO: 20;
(x) a V_{H} comprising the amino acid sequence of SEQ ID NO: 14, or a variant thereof having at least about 80% sequence identity to SEQ ID NO: 14 and a V_{L} comprising the amino acid sequence of SEQ ID NO: 20, or a variant thereof having at least about 80% sequence identity to SEQ ID NO: 20; or
(xi) a V_{H} comprising the amino acid sequence of SEQ ID NO: 15, or a variant thereof having at least about 80% sequence identity to SEQ ID NO: 15 and a V_{L} comprising the amino acid sequence of SEQ ID NO: 20, or a variant thereof having at least about 80% sequence identity to SEQ ID NO: 20.

24. The combination of claim 6, wherein the anti-TNFR2 antibody comprises:
(i) a heavy chain comprising the amino acid sequence of SEQ ID NO: 28 or a variant thereof having at least about 80% sequence identity to SEQ ID NO: 28; and a light chain comprising the amino acid sequence of SEQ ID NO: 37 or a variant thereof having at least about 80% sequence identity to SEQ ID NO: 37;
(ii) a heavy chain comprising the amino acid sequence of SEQ ID NO: 29 or a variant thereof having at least about 80% sequence identity to SEQ ID NO: 29; and a light chain comprising the amino acid sequence of SEQ ID NO: 38 or a variant thereof having at least about 80% sequence identity to SEQ ID NO: 38;
(iii) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 39 or a variant thereof having at least about 80% sequence identity to SEQ ID NO: 39;
(iv) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 40 or a variant thereof having at least about 80% sequence identity to SEQ ID NO: 40;
(v) a heavy chain comprising the amino acid sequence of SEQ ID NO: 30 or a variant thereof having at least about 80% sequence identity to SEQ ID NO: 30; and a light chain comprising the amino acid sequence of SEQ ID NO: 38 or a variant thereof having at least about 80% sequence identity to SEQ ID NO: 38;
(vi) a heavy chain comprising the amino acid sequence of SEQ ID NO: 31 or a variant thereof having at least about 80% sequence identity to SEQ ID NO: 31; and a light chain comprising the amino acid sequence of SEQ ID NO: 41 or a variant thereof having at least about 80% sequence identity to SEQ ID NO: 41;
(vii) a heavy chain comprising the amino acid sequence of SEQ ID NO: 32 or a variant thereof having at least about 80% sequence identity to SEQ ID NO: 32; and a light chain comprising the amino acid sequence of SEQ ID NO: 41 or a variant thereof having at least about 80% sequence identity to SEQ ID NO: 41;
(viii) a heavy chain comprising the amino acid sequence of SEQ ID NO: 33 or a variant thereof having at least about 80% sequence identity to SEQ ID NO: 33; and a light chain comprising the amino acid sequence of SEQ ID NO: 41 or a variant thereof having at least about 80% sequence identity to SEQ ID NO: 41;
(ix) a heavy chain comprising the amino acid sequence of SEQ ID NO: 34 or a variant thereof having at least about 80% sequence identity to SEQ ID NO: 34; and a light chain comprising the amino acid sequence of SEQ ID NO: 41 or a variant thereof having at least about 80% sequence identity to SEQ ID NO: 41;
(x) a heavy chain comprising the amino acid sequence of SEQ ID NO: 35 or a variant thereof having at least about 80% sequence identity to SEQ ID NO: 35; and a light chain comprising the amino acid sequence of SEQ ID NO: 41 or a variant thereof having at least about 80% sequence identity to SEQ ID NO: 41; or
(xi) a heavy chain comprising the amino acid sequence of SEQ ID NO: 36 or a variant thereof having at least about 80% sequence identity to SEQ ID NO: 36; and a light chain comprising the amino acid sequence of SEQ ID NO: 41 or a variant thereof having at least about 80% sequence identity to SEQ ID NO: 41.

25. The combination of claim 6, wherein the anti-PD-1 antibody is selected from the group consisting of cemiplimab, nivolumab, pembrolizumab, sintilimab, camrelizumab, toripalimab, tislelizumab, zimberelimab, prolgolimab, dostarlimab, AMG-404, balstilimab, budigalimab, cetrelimab, ezabenlimab, genolimzumab, LZM-009, nofazinlimab, penpulimab, pimivalimab, pucotenlimab, QL-1604, retifanlimab, rulonilimab, sasanlimab, SCT-I10A, serplulimab, SG-001, spartalizumab, SYSA-1802 and TY-101.

26. The combination of claim 6, wherein the anti-PD-L1 antibody is selected from the group consisting of avelumab, durvalumab, atezolizumab, KN035, CK-301, ADG-104, BCD-135, garivulimab, CBT-502, HLX-20, IMC-001, tagitanlimab, LAE-005, LP-002, MSB-2311, adebrelimab, sugemalimab and socazolimab.


SEQUENCE LISTING (41 sequences)

SEQ ID NO: 1 - protein, 5 aa, synthetic construct
DDYID

SEQ ID NO: 2 - protein, 17 aa, synthetic construct
EIYPGSGNTYYNEKFKG

SEQ ID NO: 3 - protein, 11 aa, synthetic construct
SQVYGKIAMDH

SEQ ID NO: 4 - protein, 15 aa, synthetic construct
RASESVDNSGNSFMH

SEQ ID NO: 5 - protein, 7 aa, synthetic construct
RASNLES

SEQ ID NO: 6 - protein, 9 aa, synthetic construct
QQSKEDPYT

SEQ ID NO: 7 - protein, 120 aa, synthetic construct
QVQLQQSGAELARPGTSVKLSCKASGYTFTDDYIDWVKQRTGQGLEWIGEIYPGSGNTYYNEKFKGKATLTADKSSSTAYMQLSSLTSEDSAVYFCARSQVYGKIAMDHWGQGTSVTVSS

SEQ ID NO: 8 - protein, 120 aa, synthetic construct
QVQLVQSGAEVKKPGASVKVSCKASGYTFTDDYIDWVRQATGQGLEWMGEIYPGSGNTYYNEKFKGRATMTRNTSISTAYMELSSLRSEDTAVYYCARSQVYGKIAMDHWGQGTLVTVSS

SEQ ID NO: 9 - protein, 120 aa, synthetic construct
QVQLVQSGAEVKKPGASVKVSCKASGYTFTDDYIDWVRQATGQGLEWMGEIYPGSGNTYYNEKFKGRVTLTRNTSISTAYMELSSLRSEDTAVYYCARSQVYGKIAMDHWGQGTLVTVSS

SEQ ID NO: 10 - protein, 120 aa, synthetic construct
QVQLVQSGAEVKKPGASVKVSCKASGYTFTDDYIDWVRQATGQGLEWIGEIYPGSGNTYYNEKFKGRVTMTRDKSISTAYMELSSLRSEDTAVYYCARSQVYGKIAMDHWGQGTLVTVSS

SEQ ID NO: 11 - protein, 120 aa, synthetic construct
QVQLVQSGAEVKKPGASVKVSCKASGYTFTDDYIDWVRQATGQGLEWIGEIYPGSGNTYYNEKFKGRVTMTRNTGISTAYMELSSLRSEDTAVYYCARSQVYGKIAMDHWGQGTLVTVSS

SEQ ID NO: 12 - protein, 120 aa, synthetic construct
QVQLVQSGAEVKKPGASVKVSCKASGYTFTDDYIDWVRQAPGQGLEWIGEIYPGSGNTYYNEKFKGRVTMTRDTSTSTVYMELSSLRSEDTAVYYCARSQVYGKIAMDHWGQGTLVTVSS

SEQ ID NO: 13 - protein, 120 aa, synthetic construct
QVQLVQSGAEVKKPGASVKVSCKASGYTFTDDYIDWVRQATGQGLEWIGEIYPGSGNTYYNEKFKGRVTMTRNTAISTAYMELSSLRSEDTAVYYCARSQVYGKIAMDHWGQGTLVTVSS

SEQ ID NO: 14 - protein, 120 aa, synthetic construct
QVQLVQSGAEVKKPGASVKVSCKASGYTFTDDYIDWVRQATGQGLEWIGEIYPGSGNTYYNEKFKGRVTMTADKSISTAYMELSSLRSEDTAVYYCARSQVYGKIAMDHWGQGTLVTVSS

SEQ ID NO: 15 - protein, 120 aa, synthetic construct
QVQLVQSGAEVKKPGASVKVSCKASGYTFTDDYIDWVRQAPGQGLEWMGEIYPGSGNTYYNEKFKGRVTMTRDTSTSTVYMELSSLRSEDTAVYYCARSQVYGKIAMDHWGQGTLVTVSS

SEQ ID NO: 16 - protein, 111 aa, synthetic construct
DIVLTQSPASLAVSLGQRATISCRASESVDNSGNSFMHWYQQIPGQPPKLLIYRASNLESGIPARFSGSGARTDFTLTIDPVGAVDVATYYCQQSKEDPYTFGGGTKLEIE

SEQ ID NO: 17 - protein, 111 aa, synthetic construct
DIVMTQSPDSLAVSLGERATINCRASESVDNSGNSFMHWYQQKPGQPPKLLIYRASNLESGVPDRFSGSGAGTDFTLTISSLQAEDVAVYYCQQSKEDPYTFGQGTKVEIK

SEQ ID NO: 18 - protein, 111 aa, synthetic construct
DIVMTQSPDSLAVSLGERATINCRASESVDNSGNSFMHWYQQKPGQPPKLLIYRASNLESGVPDRFSGSGSGTDFTLTISSLQAEDVAVYYCQQSKEDPYTFGQGTKVEIK

SEQ ID NO: 19 - protein, 111 aa, synthetic construct
DIVLTQSPDSLAVSLGERATINCRASESVDNSGNSFMHWYQQKPGQPPKLLIYRASNLESGVPDRFSGSGSGTDFTLTISSLQAEDVAVYYCQQSKEDPYTFGQGTKVEIK

SEQ ID NO: 20 - protein, 111 aa, synthetic construct
DIVMTQSPDSLAVSLGERATINCRASESVDNSGNSFMHWYQQKPGQPPKLLIYRASNLESGVPDRFSGSGSRTDFTLTISSLQAEDVAVYYCQQSKEDPYTFGQGTKVEIK

SEQ ID NO: 21 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 107 aa, synthetic construct [sequence omitted]

SEQ ID NO: 24 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 235 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 26 - protein, 235 aa, synthetic construct
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCTCRPGWYCALSKQEGCRLCAPLRKCRPGFGVARPGTETSDVVCKACAPGTFSDTTSSTDVCRPHRICSILAIPGNASTDAVCAPESPTLSAIPRTLYVSQPEPTRSQPLDQEPGPSQTPSILTSLGSTPIIEQSTKGG

SEQ ID NO: 27 - protein, 235 aa, synthetic construct
LPAQVAFTPYAPEPGGTCRLREYYDQTAQMCCSKCPPGQHAKVFCTKTSDTVCDSCEDSTYTQLWNWVPECLSCGSRCSSDQVETQACTREQNRICTCRPGWYCALSKQEGCRLCAQLRKCRPGFGVARPGTETSDVVCKPCAPGTFSNTTSSTDICRPHQICHVVAIPGNASMDAVCTSTSPTRSMAPGAVHLPQPVSTRSQHTQPTPAPSTAPGTSFLLPVGPSPPAEGSTGD

SEQ ID NO: 28 - protein, 450 aa, synthetic construct
QVQLQQSGAELARPGTSVKLSCKASGYTFTDDYIDWVKQRTGQGLEWIGEIYPGSGNTYYNEKFKGKATLTADKSSSTAYMQLSSLTSEDSAVYFCARSQVYGKIAMDHWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 450 aa, synthetic construct
QVQLVQSGAEVKKPGASVKVSCKASGYTFTDDYIDWVRQATGQGLEWMGEIYPGSGNTYYNEKFKGRATMTRNTSISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 450 aa, synthetic construct
QVQLVQSGAEVKKPGASVKVSCKASGYTFTDDYIDWVRQATGQGLEWMGEIYPGSGNTYYNEKFKGRVTLTRNTSISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 450 aa, synthetic construct
QVQLVQSGAEVKKPGASVKVSCKASGYTFTDDYIDWVRQATGQGLEWIGEIYPGSGNTYYNEKFKGRVTMTRDKSISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 450 aa, synthetic construct
QVQLVQSGAEVKKPGASVKVSCKASGYTFTDDYIDWVRQATGQGLEWIGEIYPGSGNTYYNEKFKGRVTMTRNTGISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 450 aa, synthetic construct
QVQLVQSGAEVKKPGASVKVSCKASGYTFTDDYIDWVRQAPGQGLEWIGEIYPGSGNTYYNEKFKGRVTMTRDTSTSTVYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 450 aa, synthetic construct
QVQLVQSGAEVKKPGASVKVSCKASGYTFTDDYIDWVRQATGQGLEWIGEIYPGSGNTYYNEKFKGRVTMTRNTAISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 450 aa, synthetic construct
QVQLVQSGAEVKKPGASVKVSCKASGYTFTDDYIDWVRQATGQGLEWIGEIYPGSGNTYYNEKFKGRVTMTADKSISTAYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 450 aa, synthetic construct
QVQLVQSGAEVKKPGASVKVSCKASGYTFTDDYIDWVRQAPGQGLEWMGEIYPGSGNTYYNEKFKGRVTMTRDTSTSTVYMELSSLRSEDTAVYYCARSQVYGKIAMDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 218 aa, synthetic construct
DIVLTQSPASLAVSLGQRATISCRASESVDNSGNSFMHWYQQIPGQPPKLLIYRASNLESGIPARFSGSGARTDFTLTIDPVGAVDVATYYCQQSKEDPYTFGGGTKLEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 218 aa, synthetic construct
DIVMTQSPDSLAVSLGERATINCRASESVDNSGNSFMHWYQQKPGQPPKLLIYRASNLESGVPDRFSGSGAGTDFTLTISSLQAEDVAVYYCQQSKEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 218 aa, synthetic construct
DIVMTQSPDSLAVSLGERATINCRASESVDNSGNSFMHWYQQKPGQPPKLLIYRASNLESGVPDRFSGSGSGTDFTLTISSLQAEDVAVYYCQQSKEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 218 aa, synthetic construct
DIVLTQSPDSLAVSLGERATINCRASESVDNSGNSFMHWYQQKPGQPPKLLIYRASNLESGVPDRFSGSGSGTDFTLTISSLQAEDVAVYYCQQSKEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 218 aa, synthetic construct
DIVMTQSPDSLAVSLGERATINCRASESVDNSGNSFMHWYQQKPGQPPKLLIYRASNLESGVPDRFSGSGSRTDFTLTISSLQAEDVAVYYCQQSKEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC